(19) United States
(12) Patent Application Publication
(10) Pub. No.: US 2022/0347363 A1
(43) Pub. Date: Nov. 3, 2022

(54) CORRELATION BETWEEN INTRAPERITONEAL PRESSURE ("IPP") MEASUREMENTS AND PATIENT INTRAPERITONEAL VOLUME ("IPV") METHODS, APPARATUSES, AND SYSTEMS

(71) Applicants: BAXTER INTERNATIONAL INC., Deerfield, IL (US); BAXTER HEALTHCARE SA, Glattpark (Opfikon) (CH)

(72) Inventors: Christian Antero Vartia, Veberod (SE); Carlos Alberto Corrales Nogales, Vernon Hills, IL (US); Joanna Rita Yanaga, Arlington Heights, IL (US); Jorge Del Castillo, Des Plaines, IL (US); Per-Olof Borgqvist, Lund (SE); Anders Wallenborg, Bjarred (SE); John Sterling Norman, Gurnee, IL (US); Jeannette Aguilar, Hoffman Estates, IL (US); Madison Rachel Hedlund, Libertyville, IL (US); Caesar Gudani, Chicago, IL (US)

(21) Appl. No.: 17/734,501

(22) Filed: May 2, 2022

Related U.S. Application Data
(60) Provisional application No. 63/182,145, filed on Apr. 30, 2021.

(51) Int. Cl. A61M 1/28

(52) U.S. Cl. CPC A61M 1/28; A61M 1/282; A61M 2205/3334; A61M 2205/3331; A61M 1/285; A61M 2205/18

(57) ABSTRACT

Methods, apparatuses, and systems for determining a correlation between intraperitoneal pressure (“IPP”) measurements and patient intraperitoneal volume (“IPV”) are disclosed. In an example, an apparatus is configured to determine a maximum fill volume of dialysate to be pumped into a peritoneal cavity of a patient. The apparatus also determines a plurality of iterations to achieve the maximum fill volume and a volume of the dialysate to be pumped for each of the iterations. For each iteration, the apparatus causes a dialysis machine to pump the dialysate to the peritoneal cavity based on the determined volume for that iteration, records IPP measurement output data from a pressure sensor, records or determines a total accumulated fill volume, and creates a data point for a personalized patient model corresponding to a correlation between the IPP measurement output data and the total accumulated fill volume.

### PRIORITY CLAIM

This application claims priority to and the benefit as a non-provisional application of U.S. Provisional Patent Application No. 63/182,145, filed on Apr. 30, 2021, the entire contents of which are hereby incorporated by reference and relied upon.

### BACKGROUND

Due to various causes, a person's renal system can fail. Renal failure produces several physiological derangements. For instance, it is no longer possible for a person with renal failure to balance water and minerals or to excrete daily metabolic load. Additionally, toxic end products of metabolism, such as urea, creatinine, uric acid and others, may accumulate in a patient's blood and tissue.

Reduced kidney function and, above all, kidney failure is treated with dialysis. Dialysis removes waste, toxins, and excess water from the body that normal functioning kidneys would otherwise remove. Dialysis treatment for replacement of kidney functions is critical to many people because the treatment is lifesaving.

One type of kidney failure therapy is peritoneal dialysis (“PD”), which infuses a dialysis solution, also called dialysis fluid or PD fluid, into a patient's peritoneal cavity via a catheter. The dialysis fluid contacts a peritoneal membrane in a patient's peritoneal cavity. Waste, toxins, and excess water pass from the patient's bloodstream, through the capillaries in the peritoneal membrane, and into the dialysis fluid due to diffusion and osmosis (i.e., an osmotic gradient occurs across the membrane). An osmotic agent in the dialysis fluid provides the osmotic gradient. Used or spent dialysis fluid is drained from the patient, removing waste, toxins, and excess water from the patient. This cycle is repeated multiple times.

There are various types of peritoneal dialysis therapies, including continuous ambulatory peritoneal dialysis (“CAPD”), automated peritoneal dialysis (“APD”), tidal flow dialysis, and continuous flow peritoneal dialysis (“CFPD”). CAPD is a manual dialysis treatment. Here, the patient manually connects an implanted catheter to a drain to allow used or spent dialysis fluid to drain from the peritoneal cavity. The patient then switches fluid communication so that the patient catheter communicates with a bag of fresh dialysis fluid to infuse the fresh dialysis fluid through the catheter and into the patient. The patient disconnects the catheter from the fresh dialysis fluid bag and allows the dialysis fluid to dwell within the peritoneal cavity, where the transfer of waste, toxins, and excess water takes place. After a dwell period, the patient repeats the manual dialysis procedure, for example, four times per day. Manual peritoneal dialysis requires a significant amount of time and effort from the patient, leaving ample room for improvement.

Automated peritoneal dialysis (“APD”) is similar to CAPD in that the dialysis treatment includes drain, fill, and dwell cycles. APD machines, however, perform the cycles automatically, typically while the patient sleeps. APD machines free patients from having to manually perform the treatment cycles and from having to transport supplies during the day. APD machines connect fluidly to an implanted catheter, to a source or bag of fresh dialysis fluid, and to a fluid drain. APD machines pump fresh dialysis fluid from a dialysis fluid source, through the catheter and into the patient's peritoneal cavity. APD machines also allow for the dialysis fluid to dwell within the cavity, providing for the transfer of waste, toxins, and excess water. The source may include multiple liters of dialysis fluid including several solution bags.

APD machines pump used or spent dialysate from the patient's peritoneal cavity, though the catheter, and to the drain. As with the manual CAPD process, several drain, fill, and dwell cycles occur during dialysis. A “last fill” may occur at the end of the APD treatment. The last fill fluid may remain in the peritoneal cavity of the patient until the start of the next treatment, or may be manually emptied at some point during the day.

Oftentimes, a clinician determines certain parameters that specify how a PD treatment is to be administered. For instance, a clinician may specify a fill volume parameter that defines an amount of dialysis fluid that is to be provided into a patient's peritoneal cavity during fill phases of a treatment cycle. A clinician may also specify a drain parameter, which defines how much used or spent dialysate (and ultrafiltration) is to be removed during drains. A clinician may further specify a dwell parameter that defines a duration of time during which the dialysis fluid is to remain in the patient's peritoneal cavity. For many treatments, a clinician may also prescribe a certain concentration of dextrose for the dialysis fluid to achieve certain treatment objectives.

While all of the above-parameters are important for a PD treatment, the fill volume parameter can be critical. If the fill volume parameter is too high, a patient can become overfilled during treatment, leading to discomfort, sleep disturbances, and/or breathing difficulties. Additionally, overfilling has been associated with hernias, fluid leakage, hydrothorax, and gastroesophageal reflux. A high fill volume may also cause reabsorption of peritoneal fluid, which significantly decreases ultrafiltration. If the fill volume parameter is too low, the PD treatment may be less effective at removing accumulated toxins. Currently, some clinicians estimate the fill volume parameter using measurements of a patient's intraperitoneal pressure (“IPP”), which is a measure of pressure in a patient's peritoneal cavity as a result of accumulated fluid and waste products. Other clinicians may estimate IPP using a patient's body mass index. Generally, a patient's IPP increases as a fluid volume increases. A fill volume may be determined as an amount of PD fluid provided to a patient's peritoneal cavity that causes the pressure to reach a certain threshold, which is generally between 10 to 20 centimeters (“cm”) H₂O (0.142 to 0.284 pounds per square inch (“psig”)) for stable adult PD patients with 2 liters of dialysate.

In PD, the IPP significantly influences ultrafiltration through increases in lymphatic reabsorption of fluid, modifications of transcapillary ultrafiltration rates, and increases in fluid absorption to adjacent tissues. Even with no fluid in the peritoneal cavity, IPP varies greatly between individuals. The intraperitoneal volume (“IPV”), which is the sum of the previous residual volume, infused volume, and ultrafiltration volume, is the main factor that affects IPP. The IPP is also associated with body size and body mass index (“BMI”), and may change based on body position and/or physical activity. In some instances, the volume of a patient's peritoneal cavity is estimated using a patient's height, age, and gender in comparison to population averages for similar individuals. The estimated volume may then be adjusted based on a measured IPP for determining a fill volume parameter for a PD treatment.

For various reasons, IPP measurements may be inaccurate. The relatively low peritoneal pressure makes IPP measurements especially challenging. Additionally in some instances, a patient or measuring equipment may be moved during a measurement, which affects IPP measurements. Even slight movements can cause IPP measurements to vary by 20 to 30%. Further, patient food and beverage consumption in the twenty-four hours leading up to a measurement can affect IPP measurement results.

A need accordingly exists for improved IPP measurement systems and methods that provide a correlation with IPV.

### SUMMARY

Example systems, methods, and apparatuses are disclosed herein for determining accurate correlations between IPP and IPV for a patient, which enables a personalized, optimized fill volume parameter to be selected for a patient. The systems, methods, and apparatuses may be used for PD onboarding of patients prior to the determination of treatment parameters. During the PD onboarding process, a patient is placed in a supine or sitting position. A midline of the patient (e.g., a ‘zero’ point of a patient's peritoneum) is identified. A pressure sensor located on an IV pole clamp is adjusted to be aligned with the patient's midline, thereby eliminating pressure components due to a height mismatch with the patient. In some embodiments, the example systems, methods, and apparatuses are configured to detect a pressure via the sensor and provide feedback until the sensor is placed at an appropriate height (e.g., when a pressure measurement is approximately 0 cmH₂O). Further, the sensor may be calibrated or zeroed to atmospheric pressure.

During PD onboarding (or other instances where a relationship between IPP and IPV for a patient is needed) after the pressure sensor is positioned, a patient's peritoneal cavity may be filled with PD fluid (e.g., dialysate) using a PD machine or bagged dialysate via gravity fill. In some instances, the patient's peritoneal cavity may be drained before filling. The patient is filled in increments. For example, a patient may be initially filled with 750 milliliters (“ml”) of fluid. The patient is then filled in 250 ml increments until 2 liters (“L”) has been pumped into the patient's peritoneal cavity. After each increment, a fluid line to the patient is clamped to remove any applied pressure from a pump, gravity fill, or PD machine. The sensor is then used to perform an IPP measurement for a known fill volume. The fluid line is then unclamped and a next fill interval occurs. The process is repeated until 2 L of dialysate, for example, is provided to the patient's peritoneal cavity. In some embodiments, multiple fill cycles are repeated to obtain additional data points. Moreover, IPP measurements may be performed during drain phases to obtain further data points.

The example systems, methods, and apparatuses obtain at least two to ten (preferably at least five or six) data points of correlations between IPP and IPV for a patient. The systems, methods, and apparatuses use these data points to create a personalized regression model (e.g., a linear regression model) specific for the patient. The systems, methods, and apparatuses may also determine lower and upper prediction intervals for the personalized patient model. The personalized model is used for determining an ideal or optimized fill volume for a patient, which removes as much waste and toxins from a patient as possible per cycle without exceeding a maximum fill volume threshold of the patient.

In some embodiments, the PD onboarding performed by the systems, methods, and apparatuses is configured to obtain IPP-IPV correlations for dialysates with different dextrose concentrations. Alternatively, the systems, methods, and apparatuses create a patient model for different dextrose concentrations using only IPP-IPV correlations for one type of dextrose. The systems, methods, and apparatuses may account for different concentrations of dextrose in dialysate using peritoneal equilibration test (“PET”) diffusion ratios and ultrafiltration volumes.

In some embodiments, the PD onboarding (or other instances where a relationship between IPP and IPV for a patient is needed) performed by the systems, methods, and apparatuses includes determining IPP-IPV correlations for different patient positions and/or activities. For example, at least one IPP-IPV correlation may be performed while a patient is sitting while another IPP-IPV correlation is performed while the patient is standing or walking. In these embodiments, the systems, methods, and apparatuses are configured to adjust the patient model such that an upper prediction interval is not exceeded for any patient position/activity.

As disclosed herein, the systems, methods, and apparatuses were bench verified by comparing results of manual IPP measurements to in-line sensor IPP measurements. The systems, methods, and apparatuses demonstrated that in-line sensor IPP measurements can be just as accurate as traditional manual IPP measurements. Accordingly, the systems, methods, and apparatuses disclosed herein use an in-line sensor to perform IPP measurements, thereby reducing potential error and increasing a speed of the PD onboarding process than if manual IPP measurements were performed.

In light of the disclosure set forth herein, and without limiting the disclosure in any way, in a first aspect of the present disclosure, which may be combined with any other aspect, or portion thereof, described herein an intraperitoneal pressure (“IPP”)-intraperitoneal volume (“IPV”) correlation system includes a fluid container containing dialysate, a dialysis machine fluidly coupled to the fluid container, an in-line pressure sensor fluidly coupled to the dialysis machine and configured to transmit IPP measurement output data, and a catheter fluidly coupling the in-line pressure sensor to a peritoneal cavity of a patient. The system also includes a computer communicatively coupled to the in-line pressure sensor. The computer includes an application stored in a memory device, which when executed by a processor of the computer, causes the computer to determine or receive an indication of a maximum fill volume of the dialysate to be pumped into the peritoneal cavity of the patient for a fill phase of a dialysis cycle, determine a plurality of iterations to achieve the maximum fill volume, and determine a volume of the dialysate to be pumped for each of the iterations. For each iteration, the application is configured to cause the dialysis machine to pump the dialysate to the peritoneal cavity of the patient based on the determined volume for that iteration, cause the dialysis machine to stop pumping the dialysate when the determined volume is reached, cause at least one clamp to be actuated to isolate pressure from the dialysis machine, record IPP measurement output data from the pressure sensor, record or determine a total accumulated fill volume, create a data point corresponding to a correlation between the IPP measurement output data and the accumulated fill volume, and release the at least one clamp to enable pumping of the dialysate for the next iteration. After the plurality of iterations are complete, the application is configured to create a patient model to provide a personalized correlation between IPP measurement output data and accumulated fill volumes up to the maximum fill volume.

In a second aspect of the present disclosure, which may be combined with any other aspect listed herein, the application is configured to after the fill phase of the dialysis cycle, cause the dialysis machine to perform a dwell phase, record periodic measurements of IPP measurement output data from the pressure sensor during the dwell phase, and create data points for the patient model corresponding to a correlation between the periodic measurements of IPP measurement output data and the maximum fill volume over a time duration corresponding to the dwell phase.

In a third aspect of the present disclosure, which may be combined with any other aspect listed herein, the application is further configured to use the data points of the patient model to determine a regression curve through the data points, and determine prediction intervals for the regression curve specifying a range in which IPP measurement output data will fall when making a new prediction.

In a fourth aspect of the present disclosure, which may be combined with any other aspect listed herein, the application is further configured to use the regression curve to determine an upper prediction limit.

In a fifth aspect of the present disclosure, which may be combined with any other aspect listed herein, the application is further configured to determine or receive information indicative of an upper IPP limit for the patient, and display in the patient model the upper IPP limit in relation to the upper prediction limit to enable a maximum fill volume for a peritoneal dialysis treatment for the patient to be determined.

In a sixth aspect of the present disclosure, which may be combined with any other aspect listed herein, the application is further configured to determine or receive information indicative of an upper IPP limit for the patient, and determine a recommended maximum fill volume range having an upper bound corresponding to an IPV at an intersection of the upper IPP limit and the upper prediction limit.

In a seventh aspect of the present disclosure, which may be combined with any other aspect listed herein, the system further includes a treatment server communicatively coupled to the application. The treatment server is configured to receive, from the application, the patient model with the regression curve, the prediction intervals, and the upper prediction limit, determine or receive information indicative of an upper IPP limit for the patient, and determine a recommended maximum fill volume corresponding to an IPV at an intersection of the upper IPP limit and the upper prediction limit.

In an eighth aspect of the present disclosure, which may be combined with any other aspect listed herein, the treatment server is configured to store the recommended maximum fill volume to an electronic treatment prescription, and transmit the electronic treatment prescription to a dialysis machine associated with the patient.

In a ninth aspect of the present disclosure, which may be combined with any other aspect listed herein, the upper IPP limit is based on at least one of a patient age, a patient height, a patient weight, a patient health, a time on dialysis, a patient position, or a patient gender.

In a tenth aspect of the present disclosure, which may be combined with any other aspect listed herein, the application is configured to adjust the patient model using information indicative of ultrafiltration accumulation in the peritoneal cavity of the patient during the dwell phase, and the information indicative of ultrafiltration accumulation in the peritoneal cavity of the patient during a dwell phase is used to determine an optimal ultrafiltration time for a PD treatment dwell phase.

In an eleventh aspect of the present disclosure, which may be combined with any other aspect listed herein, the dialysate has a first concentration of dextrose or glucose and the dialysis cycle is a first dialysis cycle, and the application is configured to perform at least one addition dialysis cycle for dialysates having at least one different concentration of dextrose or glucose to obtain additional data points for the patient model.

In a twelfth aspect of the present disclosure, which may be combined with any other aspect listed herein, the dialysis cycle is a first dialysis cycle, and the application is configured to perform at least one addition dialysis cycle to obtain additional data points for the patient model.

In a thirteenth aspect of the present disclosure, which may be combined with any other aspect listed herein, the dialysis machine is a peritoneal dialysis machine.

In a fourteenth aspect of the present disclosure, which may be combined with any other aspect listed herein, the in-line sensor is positioned at a midline of the patient.

In a fifteenth aspect of the present disclosure, which may be combined with any other aspect listed herein, the application is configured to output a pressure output from the pressure sensor to enable raising or lowering of the pressure sensor for positing at the midline of the patient.

In a sixteenth aspect of the present disclosure, which may be combined with any other aspect listed herein, the application is configured to zero the pressure sensor to atmospheric pressure.

In a seventeenth aspect of the present disclosure, which may be combined with any other aspect listed herein, the system further includes a physiological sensor configured to measure a physiological parameter of the patient. The application is configured to for each iteration, receive data indicative of the physiological parameter of the patient from the physiological sensor, and associate the physiological parameter to the corresponding data point of the patient model created during the iteration for subsequent adjustment of at least one of a regression curve, prediction intervals, or an upper prediction limit determined from the patient model.

In an eighteenth aspect of the present disclosure, which may be combined with any other aspect listed herein, the system further includes a physiological sensor configured to measure a physiological parameter of the patient. The application is configured to for each iteration, receive data indicative of the physiological parameter of the patient from the physiological sensor, and associate the physiological parameter to the corresponding patient model to address changes in intraperitoneal volume due to ultrafiltration and changes in a transport capacity specific to a peritoneal membrane of the patient.

In a nineteenth aspect of the present disclosure, which may be combined with any other aspect listed herein, the application is configured to determine the maximum fill volume by receiving information indicative of the maximum fill volume, and the application is configured to determine the plurality of iterations by receiving information indicative of the plurality of iterations.

In a twentieth aspect of the present disclosure, which may be combined with any other aspect listed herein, the computer is communicatively coupled to the dialysis machine, and the application is configured to transmit instructions to the dialysis machine to cause the dialysis machine to pump the dialysate to the peritoneal cavity of the patient and cause the dialysis machine to stop pumping the dialysate when the determined volume is reached.

In a twenty-first aspect of the present disclosure, which may be combined with any other aspect listed herein, the computer is communicatively coupled to the dialysis machine, and the application is configured to receive the total accumulated fill volume from the dialysis machine.

In a twenty-second aspect of the present disclosure, which may be combined with any other aspect listed herein, at least one of the in-line pressure sensor or the computer is integrated with the dialysis machine.

In a twenty-third aspect of the present disclosure, which may be combined with any other aspect listed herein, the application is configured to perform an integrity test by filtering the IPP measurement output data, receiving patient movement or breathing data, comparing the filtered IPP measurement output data to the patient movement or breathing data, and providing an indication of an issue with the IPP measurement output data after detecting a deviation in a correlation between the filtered IPP measurement output data and the patient movement or breathing data.

In a twenty-fourth aspect of the present disclosure, which may be combined with any other aspect listed herein, the IPP measurement output data includes a pressure signal. The application is configured to sample the IPP measurement output data to extract the recorded IPP measurement output data.

In a twenty-fifth aspect, any of the features, functionality and alternatives described in connection with any one or more of FIGS. 2 to 21 may be combined with any of the features, functionality and alternatives described in connection with any other of FIGS. 2 to 21.

In light of the present disclosure and the above aspects, it is therefore an advantage of the present disclosure to provide an in-line sensor that provides for IPP measurements during a PD onboarding procedure for a patient.

It is another advantage of the present disclosure to determine an IPP-IPV correlation for a patient to provide a personalized maximum fill volume for a prescribed PD treatment.

It is yet another advantage of the present disclosure to create a personalized patient model correlating IPP and IPV over time to ensure a maximum fill does not exceed an upper IPP limit of a patient.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a diagram of a known IPP measurement technique.

FIG. 2 is a diagram that shows how a volume of a peritoneal cavity changes during respiratory inspiration and expiration.

FIG. 3 is a diagram of an example IPP-IPV correlation system, according to an example embodiment of the present disclosure.

FIG. 4 is a diagram of the IPP-IPV correlation system of FIG. 3 communicatively coupled to a treatment server via a network, according to an example embodiment of the present disclosure.

FIG. 5 is a diagram of the IPP-IPV correlation system of FIG. 3 configured to verify in-line IPP measurement output data generated by a pressure sensor using a manual method for measuring IPP, according to an example embodiment of the present disclosure.

FIG. 6 is a diagram of a fluid coupling of the IPP-IPV correlation system shown in FIG. 5, according to an example embodiment of the present disclosure.

FIG. 7 is a flow diagram of an example procedure for preparing the IPP-IPV correlation system of FIGS. 3 to 5 to create a personalized patient model, according to an example embodiment of the present disclosure.

FIG. 8 is a flow diagram of an example procedure for performing an IPP-IPV correlation using the IPP-IPV correlation system of FIGS. 3 to 5 to create a personalized patient model, according to an example embodiment of the present disclosure.

FIG. 9 is a diagram of a display interface of an application showing information indicative of a patient model, according to an example embodiment of the present disclosure.

FIG. 10 is a diagram of the display interface of FIG. 9 showing information indicative of the patient model after a complete PD cycle, according to an example embodiment of the present disclosure.

FIG. 11 is a diagram illustrative of a protocol, at least a part of which is performed by an application to implement a PD therapy to capture IPP data points, according to an example embodiment of the present disclosure.

FIG. 12 is a diagram of graph of known personalized IPP-IPV correlations for a plurality of patients, according to an example embodiment of the present disclosure.

FIG. 13 is a diagram of a patient model, according to an example embodiment of the present disclosure.

FIG. 14 is a diagram of the patient model with an upper IPP limit, according to an example embodiment.

FIG. 15 is a diagram of a patient model where IPV is defined as a function of time during a dwell, according to an example embodiment of the present disclosure.

FIG. 16 is a diagram of a graph showing an assessment of an integrity of the IPP measurement output data from an in-line pressure sensor, according to an example embodiment of the present disclosure.

FIGS. 17 and 18 are diagrams of signal processing performed on an output of an in-line pressure sensor to determine stable IPP values, according to an example embodiment of the present disclosure.

FIG. 19 is a diagram of the system configured for CAPD, according to an example embodiment of the present disclosure.

FIG. 20 is a diagram of the system of FIG. 3 with a pressure sensor included within a PD machine, according to an example embodiment of the present disclosure.

FIG. 21 is a diagram of a PD machine including a computer with an application and a patient model, according to an example embodiment of the present disclosure.

### DETAILED DESCRIPTION

Methods, systems, and apparatuses are disclosed herein for correlating IPP measurements with an IPV of a patient to determine optimized, personalized fill volume parameters for one or more PD treatments. The methods, systems, and apparatuses are configured to determine an accurate regression model, for example, that correlates IPP to IPV for a specific patient. The methods, systems, and apparatuses may also be configured to determine an upper bound prediction limit for the model based on the determined IPP-IPV correlation for a patient. The disclosed regression model provides for the determination of a PD fill volume that maximizes the amount of waste and toxins removed per cycle while staying below pressure tolerance thresholds for the patient.

The disclosed methods, systems, and apparatuses are configured to perform automated in-line IPP measurements while a patient undergoes a PD onboarding session. The use of an in-line pressure sensor improves measurement accuracy and provides an almost instantaneous measurement compared to known manual pressure measurements. Further, in-line IPP measurements provide a truer average compared to attempting to determine a midpoint of maximum and minimum IPP manual measurements due to asymmetric patient breathing. The in-line IPP measurements are processed as a time sampled signal over time, which enables filtering and averaging to account for patient disturbances to converge upon a more accurate IPP averaged value.

The in-line pressure sensor enables multiple pressure measurements to be performed during a fill phase of a PD cycle in which dialysate or other physiological compatible fluid is pumped or permitted to flow into a peritoneal cavity of a patient. These pressure measurements, when correlated with the known volume of fluid provided to the peritoneal cavity, provide a plurality of data points for creating a regression model. In some embodiments, more than one fill cycle is performed to obtain additional data points. Additionally or alternatively, IPP pressure measurements may be performed during a dwell and/or a drain phase of the PD cycle to determine how a patient's IPP changes over time.

Disclosure is directed herein to performing IPP measurements for determining a fill volume parameter for PD treatments. It should be appreciated that any of the methods, systems, and apparatuses disclosed herein may also be used to measure IPP during a PD treatment using an in-line sensor herein. IPP measurements during treatment may be used to stop PD fluid fills when a detected IPP exceeds a threshold, lengthen PD drains, and/or change from a continuous cycling peritoneal dialysis (“CCPD”) therapy to a tidal therapy when a residual volume in a patient's peritoneal cavity exceeds a threshold. In some instances, IPP measurements that exceed a threshold may trigger an alert for the patient and/or an alert to be communicated to a clinician.

Reference is made herein to IPV. As disclosed herein, IPV refers to a fluid volume in a patient's peritoneal cavity that is in addition to a patient's residual volume after a drain. The calculation of IPV refers to a volume that accommodates dialysis fluid and ultrafiltrate. The IPV values discussed herein accordingly generally do not include a patient's residual volume.

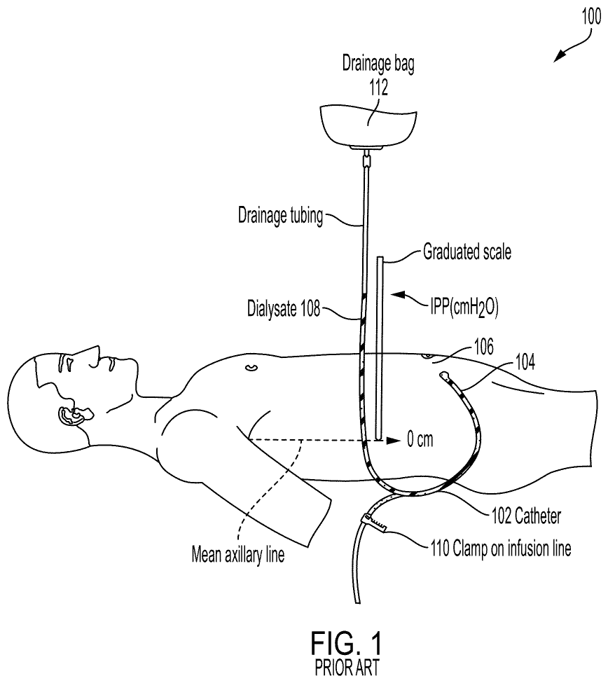

FIG. 1 shows a diagram of a known manual IPP measurement technique. A known IPP measurement system 100 includes a transfer set 102 fluidly connected to a catheter 104, which is inserted or fluidly connected to a peritoneal cavity 106 of a patient. Another end of the transfer set 102 (not shown) is connected to a source or container of fluid, such as PD fluid or dialysate. The IPP measurement system 100 also includes a measurement or drainage line 108, which is shown inverted to perform the measurement of head height. The drainage line 108 is fluidly connected to the catheter 104 and/or the transfer set 102.

An IPP measurement is performed to measure IPP in a patient's peritoneal cavity for a certain, known amount of infused PD fluid. For IPP measurements, a patient is usually positioned supine in a horizontal position, relaxed and with their head supported so the abdominal wall is relaxed to avoid pressure on the abdomen. As shown in FIG. 1, a drainage bag 112 is held in a raised support for the drainage line 108. A graduated ruler, scale, or other distance measurement device is placed next to the drainage line 108 going from the patient up to the bag 112 and aligning level 0 with a mid-axillary line (e.g., a mean axillary line), as shown.

To perform a measurement, PD fluid is provided from the source through the transfer set 102 and the catheter 104 to the peritoneal cavity 106 of the patient. The peritoneal cavity 106 is filled to a certain percentage of cavity capacity. After a desired amount of PD fluid is provided to the peritoneal cavity 106, a clamp 110 (e.g., a valve) is closed to prevent further fluid flow from the source. Next, a catheter connection is opened to enable at least some of the PD fluid from the patient's peritoneal cavity to flow into the drainage line 108. A column of the PD fluid rises in the drainage line 108 to a level where it stabilizes with a respiratory oscillation of 1 to 3 centimeters (“cm”) of H₂O, which provides an average measurement.

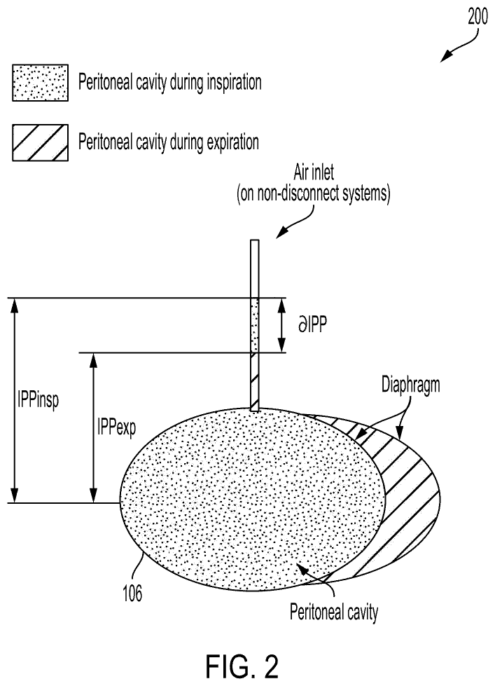

FIG. 2 is a diagram that shows how a volume of a patient's peritoneal cavity changes during respiratory inspiration and expiration. As shown in this figure, IPP during inspiration is greater because the peritoneal cavity contracts to become smaller. The IPP delta between inspiration and expiration is averaged to determine the IPP for the patient. In other words, the IPP is measured as the midpoint of that oscillation, and is expressed in centimeters of H2O. Once the measurement is obtained, the peritoneal cavity is drained and the volume is recorded in the drainage bag 112 as the fill volume. The process may be repeated for different amounts of PD fluid to determine a correlation between IPP measurements and fill volume for the particular patient.

In stable adult PD patients, an IPP of 10 to 16 cm of H₂O on the mid-axillary line is considered acceptable for PD treatments, which generally corresponds to between 1.3 and 2.8 liters (“L”) of infused PD fluid. The difference among patients between IPP and infused PD fluid volumes is due to variations in intraperitoneal volume (“IPV”), body position (with standing patients showing increases between 2 to 4 cm of H₂O compared to laying down), physical activity, weight, height, and gender. Clinicians typically prefer to keep IPP below 14 to 16 cm of H₂O since higher pressures are associated with symptoms such as discomfort, fullness, sleep disturbances, hemodynamic, and respiratory alterations. Higher pressures may also contribute to certain mechanical complications (leakage, hernia, etc.).

IPP measurements may also be made while a patient is standing or sitting. In these instances, the point “0” (e.g., the ‘zero’ point) is considered the mid-line that is located at a midpoint between the xiphoid and pubic symphysis or in the antero-superior iliac spine of the patient (e.g., the umbilicus). Despite a change in position, the IPP measurements are performed in the same manner as described above for a patient laying down.

### I. IPP Measurement Embodiments

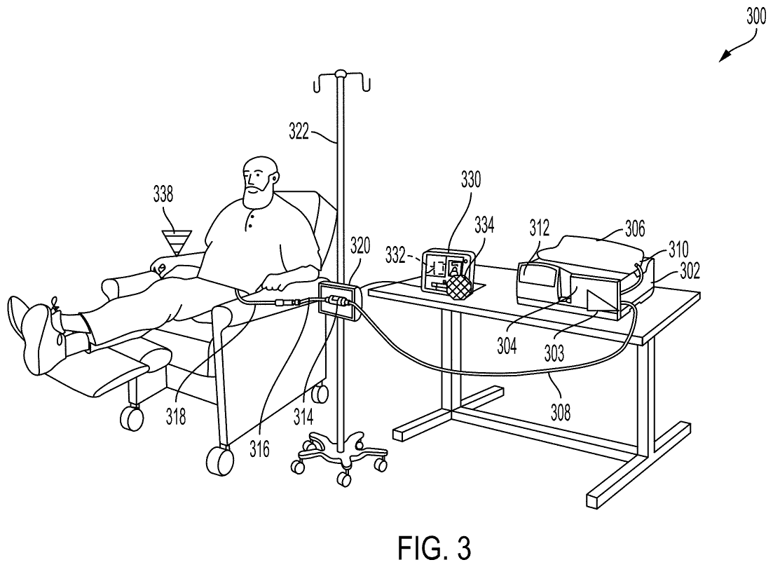

FIG. 3 is a diagram of example IPP-IPV correlation system 300, according to example embodiment of the present disclosure. The example system 300 includes a PD machine 302, a PD cassette 304, and a fluid container 306. The PD machine 302 may include any device configured to perform a PD or renal failure replacement treatment. The PD machine 302 includes one or more pumps, valves, actuators, etc. that operate in connection with the PD cassette 304 to move fluid from the fluid container 306 to a first fluid line 308. The PD cassette 304 may be disposable and may include one or more flexible fluid chambers and/or tubes to provide a fluid connection between the fluid container 306 and the first fluid line 308. The PD cassette 304 may include a rigid frame to support the flexible fluid chambers, internal fluid pathways, and/or tubes. In some embodiments, the PD cassette 304 may be omitted or integrated with the PD machine 302 for pumping fluid from the fluid container 306.

As mentioned above, the PD machine 302 includes at least one pump 303. The pump 303 may include a pump head that is fluidly connected to the first fluid line 308 via the PD cassette 304. The pump 303 may be any type of fluid pump, such as a peristaltic pump, a piston pump, a gear pump, or a membrane pump. The pump head may be part of the PD cassette 304 that is connected to a reusable actuator, which is controlled by an internal or external processor. The example pump 303 is configured to pump fresh fluid from the container 306 to the patient's peritoneal cavity to deliver dialysis fluid, enabling IPP measurements to be performed. The example pump 303 may also pump used PD fluid (including removed toxins and absorbed ultrafiltration) from the patient's peritoneal cavity back to a drain container or a drain (not shown) after a dwell phase has ended. In alternative embodiments, separate pumps are provided for (i) pumping fluid to a patient and (ii) pumping or pulling fluid from the patient. In some embodiments, the pump is configured to occlude fluid flow from the fluid container 306 until the pump head is actuated, thereby preventing free flow of PD fluid, thereby enabling a clamp to be omitted.

The fluid container 306 includes, for example, a bag of dialysate, PD fluid, renal failure replacement fluid, saline, or other physiologically compatible fluid. In an example, the fluid container 306 may contain Dianeal® Low Calcium with 2.5% dextrose or Dianeal® Low Calcium with 4.25% dextrose. In some instances, the fluid container 306 may have a rigid housing. A scale 310 may be integrated with the PD machine 302 and configured to record a weight of the fluid container 306. Alternatively, the scale 310 may be separate from the PD machine 302 and positioned to weigh the fluid container 306 and/or a drain container. A decrease in weight during the PD onboarding session is indicative of an amount of fluid pumped into a peritoneal cavity of a patient.

The example PD machine 302 may also include a control interface 312, such as a touchscreen interface. The control interface 312 is configured to receive clinician (or patient) inputs for programming a PD treatment or a PD onboarding session. The control interface 312 is communicatively coupled to a processor and a memory device of the PD machine 302. Instructions stored in the memory device are executable by the processor to enable the PD machine 302 and/or the control interface 312 to perform the operations described herein. In some instances, the PD machine 302 may include a network transceiver to enable communication with a network, such as the Internet, a cellular network, a Wi-Fi network, or combinations thereof.

The first fluid line 308 may include a transfer set or any other tubing formed from polyvinyl chloride (“PVC”), polyethylene (“PE”), polyurethane (“PU”), polycarbonate, or other non-PVC material. The first fluid line 308 is fluidly coupled to a pressure sensor 314, which may include the Deltran I Pressure Transducer produced by Utah Medical®. In other embodiments, the sensor 314 may include any other in-line sensor for measuring fluid pressure. The sensor 314 may include a piezoresistive strain gauge, a pressure sensing diaphragm, a capacitive diaphragm, a pressure sensing capsule, and/or a bourdon tube. Further, the sensor 314 may be disposable or durable.

When PD fluid is provided to the peritoneal cavity or removed from the peritoneal cavity, pressure measurements performed by the pressure sensor 314 are indicative of fluid pressure delivered to or removed from the peritoneal cavity. When pumping stops and the PD fluid is permitted to dwell in the peritoneal cavity for a specified duration, the pressure measurements recorded by the pressure sensor 314 are indicative of IPP. The pressure measurements may also be used for detecting a line occlusion (based on an upward positive or negative pressure spike/trend) or a fluid leak (based on downward positive or negative pressure spike/trend).

In the illustrated example, the pressure sensor 314 is shown as being in-line with a transfer set 318 and the fluid lines 308 and 316. It should be appreciated that the pressure sensor 314 may be in-line or otherwise integrated with a catheter in other embodiments. It should also be appreciated that the pressure sensor 314 may include disposable tube sections that contact the transfer set 318 (or the fluid line 316) and the PD fluid, while the remainder of the sensor 314 is reusable between IPP measurements. Alternatively, the entire pressure sensor 314 may be disposable.

An opposite end of the IPP measurement sensor 314 is fluidly coupled to the second fluid line 316, which may include any type of transfer set or tubing. The second fluid line 316 is fluidly coupled to a patient via the PD transfer set 318, which may include one or more catheters. The PD transfer set 318 includes an end that is connected to the catheter, which is inserted into a peritoneal cavity of the patient. Together, the fluid container 306, the PD cassette 304, the first fluid line 308, the pressure sensor 314, the second fluid line 316, and the PD transfer set 318 form a fluid pathway to the peritoneal cavity of the patient.

In the illustrated example of FIG. 3, the pressure sensor 314 is connected to a housing 320, which is mechanically connected to a pole 322 (e.g., an IV pole). The housing 320 includes a clamp that enables the housing 320 be slide along a height of the pole 322. In some embodiments, the pressure sensor 314 may be integrated into the housing 320. The housing 320 is slidable along the pole 322 to enable a height of the sensor 314 to be changed relative to the patient. As discussed below, the housing 320 is lowered or raised such that the sensor 314 is provided on a same plane as a midline of the patient (e.g., a mid-axillary line), thereby eliminating pressure measurement components caused from a height difference between the sensor 314 and the patient's peritoneal cavity.

The example IPP-IPV correlation system 300 of FIG. 3 also includes a computer 330 (e.g., a monitor) that is communicatively coupled to the sensor 314. The computer/monitor 330 may include any computer, laptop, workstation, tablet computer, server, etc. In some embodiments, the computer/monitor 330 is communicatively coupled to the pressure sensor 314 via a wired interface, such as a universal serial bus (“USB”) connection, or a wireless interface, such as a Bluetooth®, a Zigbee®, or a Near-Field Communication (“NFC”) connection. Further in some embodiments, the computer/monitor 330 may also be communicatively coupled to the PD machine 302.

As described herein, the example computer/monitor 330 executes machine readable instructions stored in a memory device. The instructions may comprise an application or software program 332. Execution of the instructions causes the computer/monitor 330 to perform the operations described herein. For instance, the computer/monitor 330 receives IPP measurement output data, which is transmitted from the pressure sensor 314. The computer/monitor 330 may also receive fill volume data and/or weight data input by a clinician or transmitted from the PD machine 302. As disclosed herein, the computer/monitor 330 is configured to use the application 332 to correlate the IPP measurement output data with the fill volume data to create a data model 334 (e.g., an upper prediction limit determined from a regression model) for a patient.

In some embodiments, the computer/monitor 330 is configured to determine upper and lower prediction limits for the model 334 and/or determine one or more fill volumes for a personalized PD treatment for the patient. The operations performed by the computer/monitor 330 accordingly provide for the determination of a fill volume parameter for PD treatments. In some embodiments, the computer/monitor 330 uses the received data to calculate or otherwise determine a fill volume parameter for a patient under measurement. Additionally or alternatively, the computer/monitor 330 may cause a display device to display a correlation of IPP measurements to fill volumes via a regression model to enable a clinician to determine a fill volume parameter for a patient's PD treatment. For brevity, the computer/monitor 330 is referred to herein as a computer 330.

In some embodiments, the application 332 on the computer 330 is configured to operate in connection with the sensor 314 to determine an appropriate height along the pole 322. In these embodiments, the fluid lines 308 and 316 may be primed with dialysate before or after a height for the pressure sensor 314 is determined. The computer 330 receives IPP measurement output data from the sensor 314. When the IPP measurement output data is greater than a specified threshold, the application 332 determines that the sensor 314 is not aligned with a midline of the patient. The application 332 accordingly causes the computer 330 to display a prompt for a clinician to increase a height of the sensor 314. In some instances, the application 332 is configured to determine a height that the sensor 314 is to be raised on the pole 322 via the housing 320 based on the IPP measurement output data. For instance, a negative cmH₂O may indicate that the sensor 314 is to be moved upward along the pole 322. The application 332 is configured to receive subsequent IPP measurement output data from the sensor 314 and continue providing prompts to raise or lower the sensor 314 until the IPP measurement output data is equal to or approximately 0 cmH₂O.

In some embodiments, the system 300 of FIG. 3 may include a line clamp (not shown) to selectively restrict the flow of fluid through the first fluid line 308. Alternatively, the PD machine 302 may include a valve or line clamp to selectively restrict the flow of fluid through the first fluid line 308. The clamp is actuated during an IPP pressure measurement to prevent the PD machine 302 for affecting a measured pressure. The clamp is located at a same or lower level compared to the sensor 314 to prevent additional head height from impacting the IPP measurement.

In some embodiments, the IPP measurements are configured to be performed a certain time after clamping. The time is chosen to enable the patient's peritoneum to stabilize after being at least partially filled with dialysate. The time may be at least two seconds and up to one or a few minutes.

In some embodiments, the system 300 may also include a flow sensor (not shown) to measure a volume of PD fluid provided to the patient and/or removed from the patient. It should be appreciated that one or more pressure sensors 314 may additionally or alternatively be used to measure a flow or flow rate of the fluid delivered to or removed from the peritoneal cavity by calculating the pressure difference between the two sensors. Further, the system 300 may include a heater for warming the PD fluid prior to infusion into the patient. The system 300 may further include a temperature sensor to ensure the PD fluid is heated to a desired temperature.

Although not illustrated, an airtrap may be provided in the first fluid line 308, the pressure sensor 314, and/or the second fluid line 316. The airtrap is configured to remove air from the PD fluid prior to patient delivery. In other instances, priming of the first fluid line 308 and the second fluid line 316 may remove air without the need for an airtrap. Heating dialysis fluid tends to separate dissolved air from the dialysis fluid. It is accordingly contemplated to locate the airtrap downstream from a heater, e.g., along the first fluid line 308 and upstream of a temperature sensor.

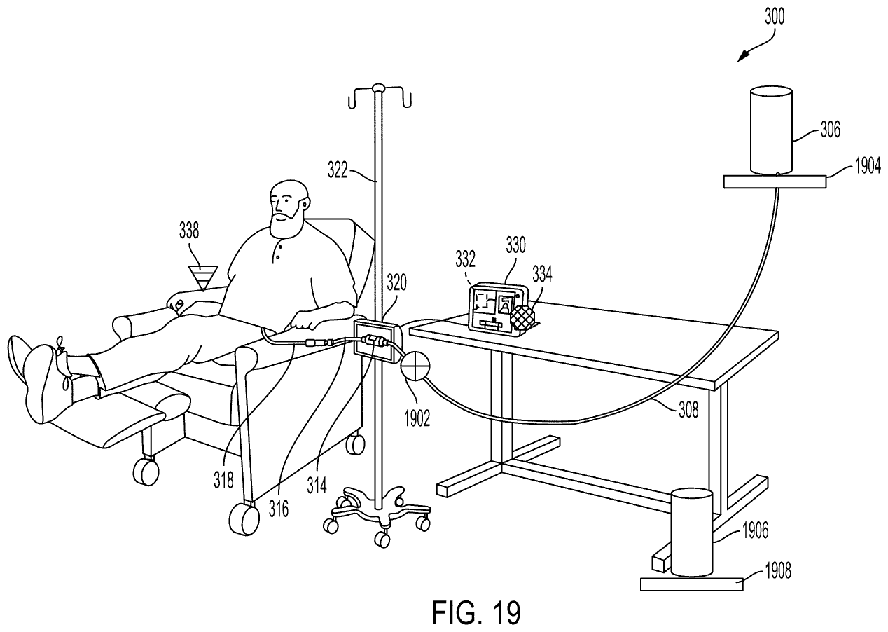

In alternative embodiments, the PD machine 302 of FIG. 3 may be replaced by positioning the fluid container 306 at or above a head-height of a patient (e.g., three to six feet above ground level) (e.g., see FIG. 19). This enables gravity to pull PD fluid from the fluid container 306 through the first fluid line 308, the second fluid line 316, and the PD transfer set 318 to the peritoneal cavity of the patient. In the illustrated example, a clamp provides for selective flow of the PD fluid.

The example system 300 of FIG. 3 may also include one or more physiological sensors 338 configured to monitor a physiological parameter of the patient or dialysis fluid. The physiological sensors 338 may include a blood pressure sensor or cuff, a heart rate sensor, a weight sensor, a respirator, etc. In some embodiments, the sensor 338 may be included in a personal device, such as a smartwatch or smart eyewear or integrated with the computer 330 or the PD machine 302. The physiological sensors 338 are communicatively coupled to the computer 330 via a wired or wireless connection including USB or Bluetooth®. Physiological data measured by the one or more sensors 338 is transmitted to the computer 330, which may use the data in connection with the IPP-IPV correlation to determine an optimal fill volume and/or fill thresholds of a patient. The physiological data may also include a patient's reported tolerance or comfort level on a scale (e.g., 1 to 5), which in input into the application 332. In an example, the computer 330 is configured to compare heart rate and/or blood pressure to a fill volume. The computer 330 determines that fill volumes greater than 1850 mL, for example, correspond to an increase in patient blood pressure and/or heart rate. The computer 330 is configured to recommend and/or limit an upper fill limit to around 1850 mL for a regression-based patient model 334 using the physiological data and/or the upper prediction limit.

In some embodiments, the application 332 of the system 300 is configured to monitor for high or low IPP measurement output data values. An upper testing threshold may include 20 or 25 cmH₂O while a lower threshold may include 1 or 5 cmH₂O. In some embodiments, the application 332 only applies the lower testing threshold after subsequent fill volumes in a patient cause the IPP measurement output data to exceed the lower testing threshold. Detection of the IPP measurement output data exceeding the upper or lower testing thresholds causes the application 332 to transmit or display an alarm or an alert. The application 332 may also cause the filling of the patient with dialysate of PD fluid to pause or stop until the pressure is within the testing thresholds. The alarms or alerts may be indicative of an issue with collection of the IPP measurement data such as a line occlusion or a fluid leak in the fluid connections shown in the system 300 of FIG. 3.

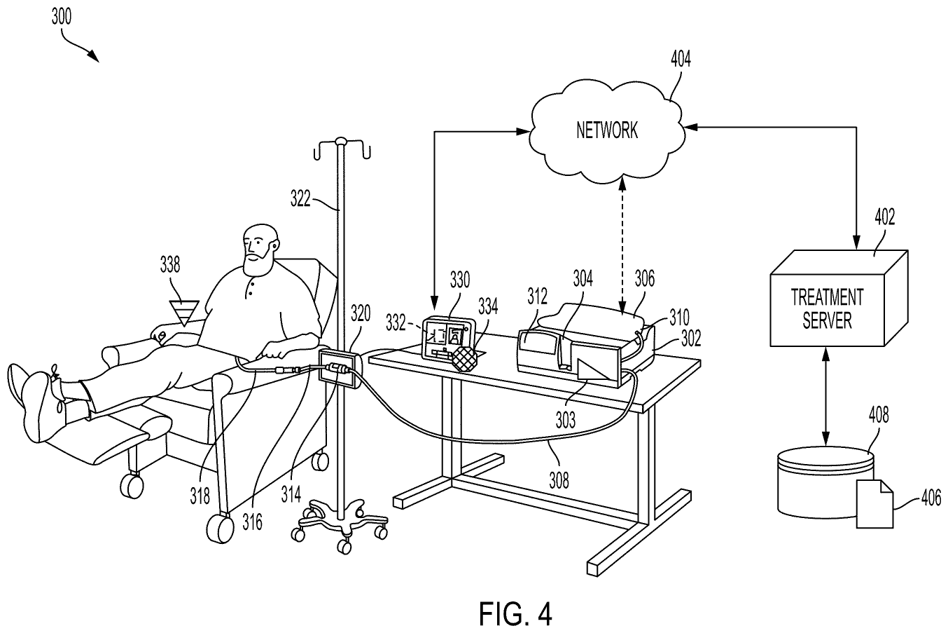

FIG. 4 is a diagram of the IPP-IPV correlation system 300 of FIG. 3 communicatively coupled to a treatment server 402 via a network 404, according to an example embodiment of the present disclosure. The network 404 may include any wide area or local network including, for example, the Internet, a cellular network, a Wi-Fi network, or combinations thereof. The treatment server 402 may include any processor, computer, controller, workstation, distributed computing network, etc. The treatment server 402 may be located within a hospital or clinical health system. Alternatively, the treatment server 402 may be a clinician computer, and may include a smartphone, a tablet computer, a workstation, a laptop, or a desktop computer.

In the illustrated embodiment, at least the computer 330 is connected to the network 404. In some embodiments, the PD machine 302 may also be connected to the network 404. Further, the physiological sensors 338 or other medical devices associated with the patient may be connected to the treatment server 402 via the network 404.

The example computer 330, in some examples, is configured to create a patient model 334 based on a correlation of IPP-IPV data points. In these examples, the computer 330 transmits the patient model 334 to the treatment server 402. In some instances, the treatment server 402 uses the upper prediction limit alone or in conjunction with a known weight, gender, height, health, etc. of the patient to estimate an IPP upper limit (e.g., a value between 14 to 16 cmH₂O). In other instances, the treatment server 402 may receive the IPP upper limit from a clinician. The treatment server 402 displays the patient model 334 with the IPP upper limit to enable an optimal patient fill volume to be selected. The optimal fill volume corresponds to a maximum volume of fluid that corresponds to an IPP below (e.g., slightly below) the IPP upper limit (e.g., between 14 to 16 cmH₂O). Such a fill volume minimizes IPP from inhibiting waste and fluid removal during a PD cycle without causing adverse patient affects or reabsorption of the waste and toxins. The optimized fill volume may also provide for fewer therapy cycles and a lower therapy time. In some embodiments, the treatment server 402 may determine and display a personalized fill volume recommendation for the patient using the IPP upper limit and the patient model 334.

As shown in the illustrated example, the PD fill volume is stored to an electronic treatment prescription 406. The example electronic treatment prescription 406 may also store other PD parameters, such as a dwell duration, a number of cycles per PD treatment, a number of treatments per day, a number of treatments per week, a PD fluid dextrose concentration, a target UF removal rate, etc. The treatment server 402 is configured to store the electronic treatment prescription 406 to a memory device 408, which may include any solid state, flash, or hard disk drive.

In some embodiments, the treatment server 402 is configured to transmit the electronic treatment prescription 406 to the PD machine 302 via the network 404. The electronic treatment prescription 406 is used by the PD machine 302 to perform a PD or other renal failure replacement treatment. In some embodiments, the treatment server 402 is instructed to transmit the electronic treatment prescription 406 to another PD machine that is located in the patient's residence and assigned to the patient.

In some alternative embodiments, the PD fill volume may be determined by the computer 330 or input into the computer 330. In these alternative embodiments, the computer 330 is configured to transmit the PD fill volume to the treatment server 402 via the network 404. The treatment server 402 is configured to store the received PD fill volume to the electronic treatment prescription 406.

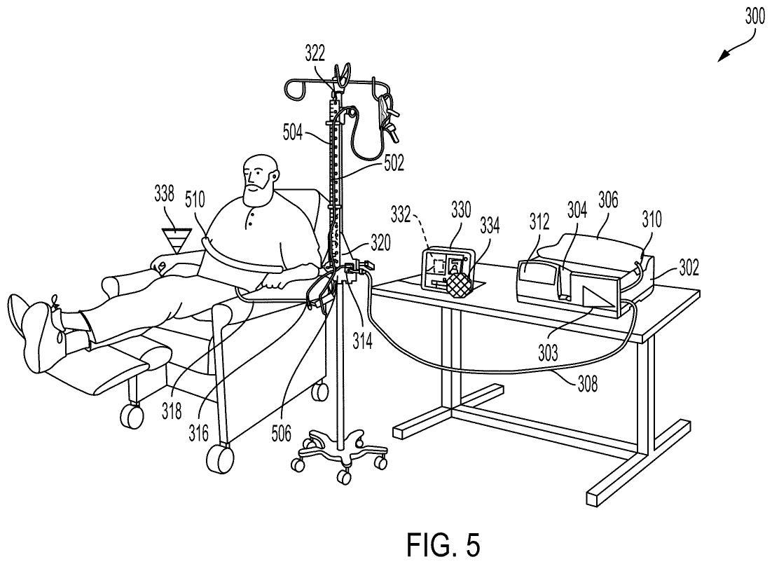

FIG. 5 is a diagram of the IPP-IPV correlation system 300 of FIG. 3 configured to verify in-line IPP measurement output data generated by the pressure sensor 314, according to an example embodiment of the present disclosure. In this example, a measurement fluid line 502 is connected to the pole 322. Additionally, the pole 322 includes a ruler 504 to provide for manual measurement of a solution column in the measurement fluid line 502 in cmH₂O, as discussed above in connection with FIG. 1. Further, a three-way connector 506 is fluidly coupled between the measurement fluid line 502 and the second fluid line 316.

In this example, at each measurement interface, the pressure sensor 314 is configured to perform an in-line IPP measurement. At the same time, a fluid level is measured in the measurement fluid line 502 with respect to the ruler 504 to perform a manual IPP measurement. The in-line sensor measurement is compared to the manual IPP measurement to verify an accuracy of the in-line IPP measurement, as discussed below.

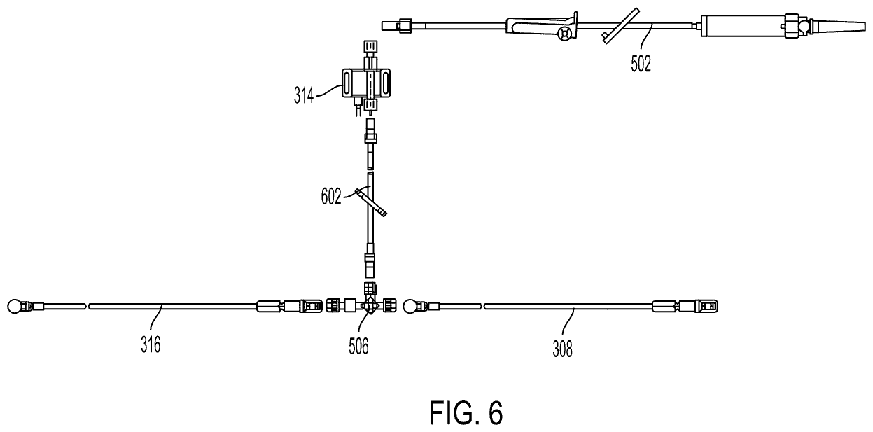

FIG. 6 is a diagram of the fluid coupling of the IPP-IPV correlation system 300 shown in FIG. 5, according to an example embodiment of the present disclosure. In this example, the measurement fluid line 502 optionally includes a Clearlink® system solution set with a DUOVENT spike. A filter circuit of the DUOVENT spike is used as a vent while the fluid circuit is closed to atmosphere by being capped with a non-vented spike cap. The pressure sensor 314 is connected in-line with the measurement fluid line 502. An extension set 602 is connected between the pressure sensor 314 and the three-way connector 506. One end of the three-way connector 506 is fluidly coupled to the first fluid line 308 and an opposite end of the three-way connector 506 is fluidly coupled to the second fluid line 316.

Returning to FIG. 5, a center of the pressure sensor 314 is positioned to align with a zero point of the ruler 504. This alignment ensures the in-line sensing and the manual IPP measurements are measuring fluid pressure from a same reference point. In some instances, an electrical output of the pressure sensor 314 is amplified prior to be received in the computer 330. The application 332 is configured to convert the amplified electrical output into digital IPP measurement output data, which is recorded as a data point for creating the patient model 334.

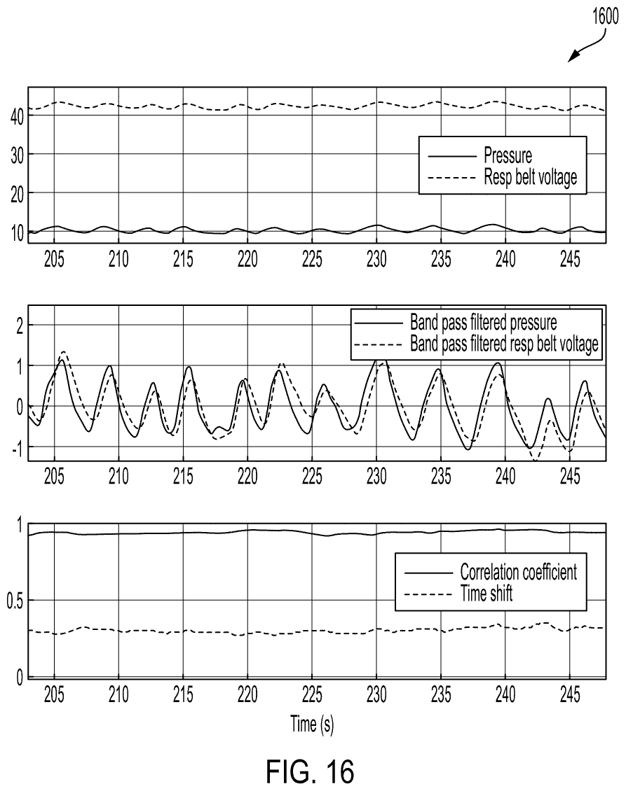

FIG. 5 also shows a respiratory belt 510 connected to a waist of a patient. The respiratory belt 510 includes at least one sensor for detecting patient movement and/or breathing. The sensor may include a piezo-resistive sensor element, a capacitive sensor element, and/or an inertial sensor element for measuring pressure and/or movement. In some embodiments, a data output from the respiratory belt 510 is received in the computer 330 and used for selecting or averaging the IPP measurement output data. For example, IPP measurement output data may be received during different portions of a breathing cycle. The application 332 is configured to average the IPP measurement output data to adjust for variation during patient breathing, which can cause as much as 2 cmH₂O of variation. The respiration signal received from the respiratory belt 510 may also be correlated with the pressure transducer signal from the sensor 314 to verify the measurement system integrity, as discussed below in connection with FIG. 16.

A comparison of in-line sensor and manual IPP measurements was performed during a bench verification. The bench verification showed there is only a small difference between in-line sensor measurements and manual IPP measurements over six cycles of at least two different runs. During the cycles, different amounts of dialysate were pumped into a chamber representative of a patient's peritoneal cavity to produce different average pressures. The minimal difference between manual and in-line sensor measurements indicates that the pressure sensor 314 may be used to record IPP-IPV correlations during a PD onboarding process.

### II. Example IPP-IPV Correlation Procedures

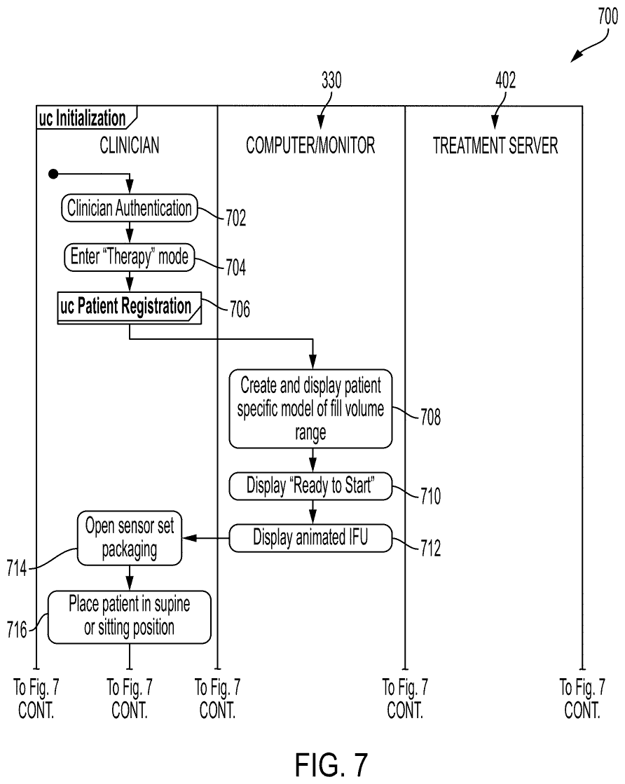

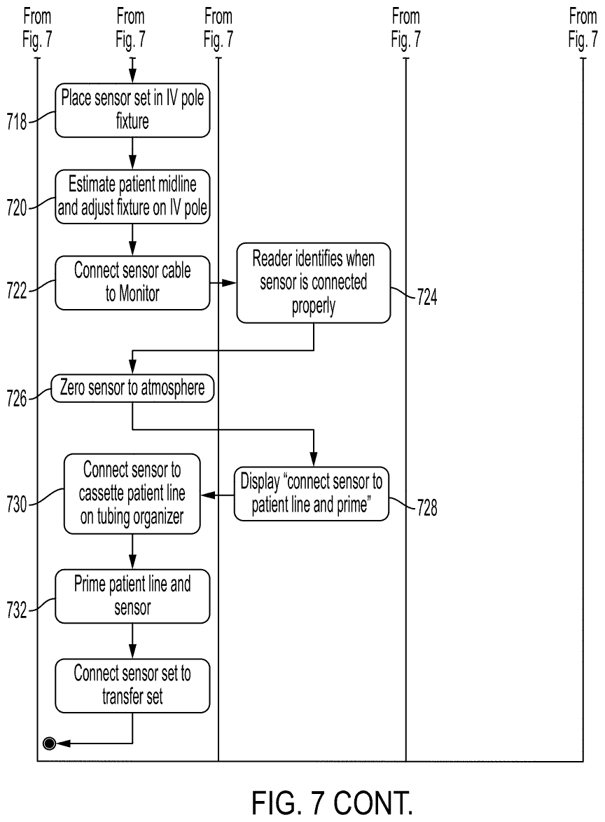

FIG. 7 is a flow diagram of an example procedure 700 for preparing the IPP-IPV correlation system 300 of FIGS. 3 to 5 to create a personalized patient model 334, according to an example embodiment of the present disclosure. Although the procedure 700 is described with reference to the flow diagram illustrated in FIG. 7, it should be appreciated that many other methods of performing the steps associated with the procedure 700 may be used. For example, the order of many of the blocks may be changed, certain blocks may be combined with other blocks, and many of the blocks described may be optional. In an embodiment, the number of blocks may be changed based, for example, on whether the estimation of a height location of the sensor 314 is automated by the computer 314. The actions described in the procedure 700 are specified by one or more instructions and may be performed among multiple devices including, for example, the pressure sensor 314, the computer 330 (e.g., the application 332), the PD machine 302, and/or the treatment server 402.

The example procedure 700 begins when a clinician begins a new session on the application 332 for PD onboarding of a patient (block 702). The clinician uses the application 332 to select a therapy mode (block 704). The clinician may also specify a therapy mode on the PD machine 302. Additionally, the clinician performs a patient registration (block 706). Registration may include obtaining or otherwise entering a patient gender, age, height, weight, health, etc. In some embodiments, registration may include accessing patient information from a medical record stored in the memory device 408.

The example application 332 next creates an initial patient model 334 using at least some of the registration information (block 708). In some embodiments, initial creation of the patient model may be omitted if the patient model 334 is created using primarily the IPP measurement output data. Instead of a patient model, the application 332 may display an estimated fill volume range for the PD onboarding using the information from the patient registration. The fill volume range may be between, for example, 1850 mL and 2250 mL for a mid-aged adult male. After confirmation by the clinician, the application 332 provides an indication that setup can begin (block 710). The application 332 may then display an animated or graphical set up procedure (“IFU”) to guide a clinician through the onboarding setup (block 712).

In some embodiments, the application 332 uses patient population models that correspond to a weight, age, gender, etc. of a current patient to determine a maximum fill volume of dialysate to be pumped into a peritoneal cavity of the patient for the PD onboarding process. Alternatively, the application 332 receives a selection of a maximum fill volume from a clinician. The application 332 then determines a plurality of iterations to achieve the maximum fill volume and a volume of the dialysate to be pumped for each of the iterations. The determinations may be made based on specified conditions. For example, the conditions may specify that a first iteration should provide 30% to 50% of a maximum fill volume and subsequent iterations should provide 10% to 15% increments of the maximum fill volume.

A first set of the onboarding setup includes causing the clinician to open a sensor set packaging for the pressure sensor 314, the fluid lines 308, 316, and/or the PD transfer set 318 (block 714). Further, the application 332 prompts the clinician to place the patient in a supine or sitting position (block 716). The application 332 then prompts the clinician to fluidly connect the fluid lines 308 and 316, the sensor 314, and the PD transfer set 318 together (block 718). This also includes connecting the sensor 314 to the housing 320 on the pole 322. In some embodiments, the clinician identifies the patient's midline and accordingly aligns the sensor 314 with the midline.

After the sensor 314 is positioned, the application prompts the clinician to connect the pressure sensor 314 to the computer 330 (block 722). This may include making a connection via a wire, such as a USB or serial cable. This may also include making a wireless connection using, for example, a Bluetooth® pairing procedure. If the application 332 is configured to provide for sensor 314 alignment, the application 332 receives IPP measurement output data from the sensor 314 (block 724). The application 332 may calculate a position for the sensor 314 based on deviations from the IPP measurement output data from a value of 0 cmH₂O. In other embodiments, the application 332 displays the IPP measurement output data, and a clinician adjusts a height of the sensor 314 (via the housing 320 connected to the pole 322) until the IPP measurement output data has a value of approximately 0 cmH₂O. The application 332 may also zero the pressure sensor 314 to atmosphere (block 726). This step may be performed after the height adjustment of the sensor 314 achieves a value that is close to 0 cmH₂O.

The example procedure 700 of FIG. 7 continues by the application 332 instructing the patient to connect the pressure sensor 314 and the first fluid line 308 to the second fluid line 316 (block 728). The application 332 also provides a prompt instructing the clinician to connect the first fluid line 308 to the PD cassette 304, which may be connected to the fluid container 306 (block 730). The application 332 may then cause the PD machine 302 (or prompt a clinician to begin a gravity fill) to perform a priming procedure where fluid is pumped from the fluid container 306 to an end of the second fluid line 316 (block 732). After priming, the application 332 prompts the clinician to connect the second patient line 316 to the PD transfer set 318 to provide a fluid connection to the peritoneal cavity of the patient (block 732). At this point, the example system 300 is ready to perform IPP-IPV correlation measurements and the procedure 700 ends.

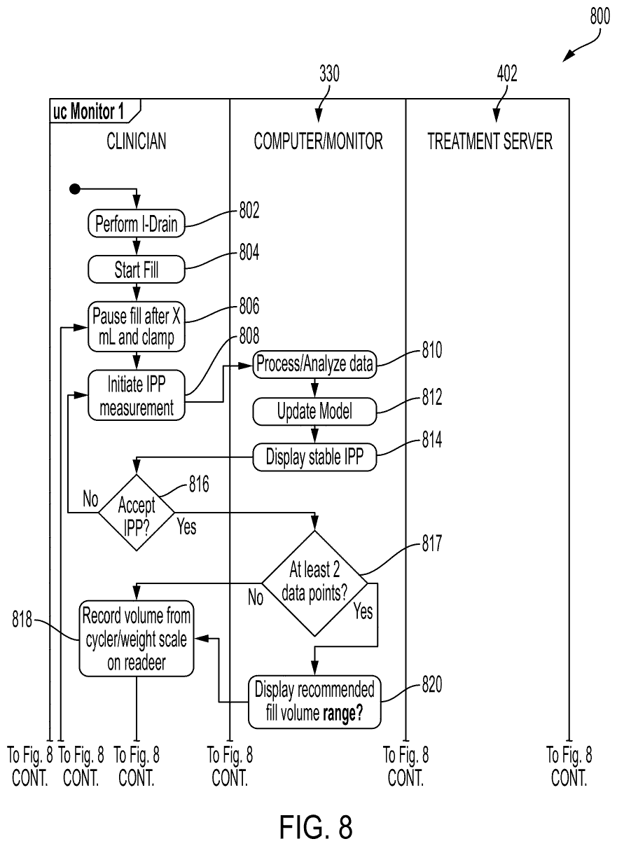

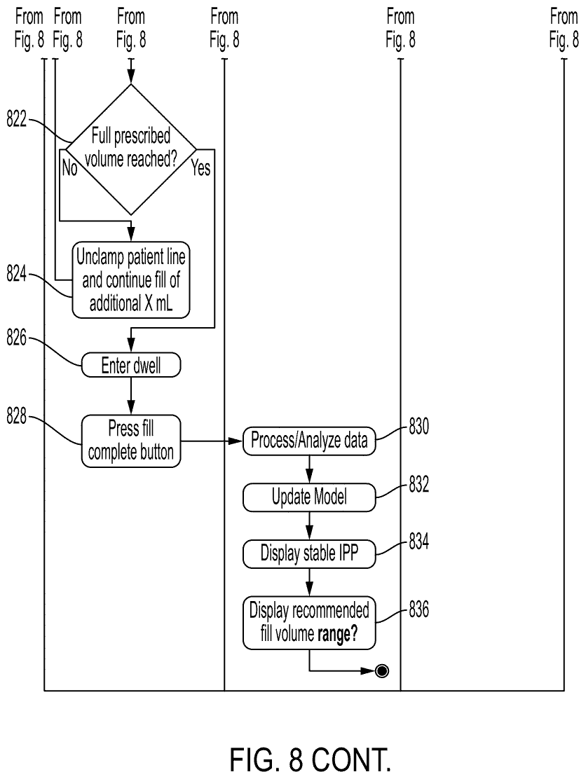

FIG. 8 is a flow diagram of an example procedure 800 for performing an IPP-IPV correlation using the IPP-IPV correlation system 300 of FIGS. 3 to 5 to create a personalized patient model 334, according to an example embodiment of the present disclosure. Although the procedure 800 is described with reference to the flow diagram illustrated in FIG. 8, it should be appreciated that many other methods of performing the steps associated with the procedure 800 may be used. For example, the order of many of the blocks may be changed, certain blocks may be combined with other blocks, and many of the blocks described may be optional. In an embodiment, the number of blocks may be changed based on a number of cycles performed, a number of intervals during each cycle, and/or whether separate cycles are performed for dialysates with different dextrose concentrations. The actions described in the procedure 800 are specified by one or more instructions and may be performed among multiple devices including, for example, the pressure sensor 314, the computer 330 (e.g., the application 332), the PD machine 302, and/or the treatment server 402.

The example procedure 800 begins after the procedure 700 ends when a patient is fluidly coupled to the PD machine 302. The procedure starts when the application 332 on the computer 330 prompts a clinician or transmits an instruction to the processor of the PD machine 302 to perform an initial patient drain (block 802). The application 332 then prompts the clinician or transmits an instruction to the processor of the PD machine 302 start a fill of the patient's peritoneal cavity with dialysate via the MD machine 302 (block 804). A first fill iteration is reached after a certain initial fill volume is provided to the patient (block 806). At this point, the application 332 prompts the clinician or transmits an instruction to the processor of the PD machine 302 to pause the fill phase. Additionally, the application 332 prompts the clinician or transmits an instruction to the processor of the PD machine 302 to close a clamp or cause the PD machine 302 to actuate an internal clamp to isolate pressure from the PD machine 302. The application 332 then performs an initial IPP measurement by recording IPP measurement output data from the pressure sensor 314 (block 808).

The application 332 then filters, removes noise, or otherwise processes the IPP measurement output data (block 810). In some instances, the application 332 may average the IPP measurement output data over breathing cycles of the patient as part of the analysis. This averaging provides a stable IPP measurement (block 814). Additionally or alternatively, the application 332 analyzes a stream of IPP measurement output data to determine when the pressure measurements have stabilized. The application 332 then uses the IPP measurement output data after detected stabilization for the IPP-IPV correction.

In some embodiments, the application 332 is configured to permit the clinician to accept the IPP measurement output data (block 816). If the clinician believes the measurement may not be accurate due to patient movement, a fluid leak, a line occlusion, etc., the clinician may provide an input to the application 332 causing the IPP measurement output data to be disregarded. The application 332 then performs another IPP measurement (block 808). However, when there is an acceptance of the IPP measurement output data, the application 332 may determine if at least two data points have been acquired for different fill volumes (block 817). If less than two data points have been collected, the application 332 prompts the clinician to enter a fill volume (block 818). In some instances, the application 332 receives the fill volume, a current weight of the fluid container 306, or a fill rate and fill time from the PD machine 302. If a fill weight is used to track a fill volume, the application 332 is configured to determine an initial fill weight of the fluid container 306 and subtract the current weight to determine a weight of fluid provided to the patient (accounting for a volume in the fluid lines 308 and 316, the transfer set 318, and the PD cassette 304). The weight of the fluid is then converted into a volume using a known density of the fluid. If a fill rate and time is used to track fluid volume, the application 332 is configured to multiply a fill rate by a volume of fluid pumped per second. This result is then multiplied by the fill time to determine a total fill volume (accounting for a volume in the fluid lines 308 and 316, the transfer set 318, and the PD cassette 304). The application 332 then creates a correlation between the IPP measurement output data and the volume of fluid (e.g., the IPV), which is stored to the patient model 334. The IPP-IPV correlation may also include a reference to a patient position, such as standing, sifting, supine, etc. If there are at least two IPP-IPV correlation data points for the model 334, the application 332 is configured to update a recommended fill volume range (block 820). Determining the recommendation may include determining a linear regression of the data points, determining a prediction limit for the linear regression, and/or applying an IPP upper limit based on patient demographic information. The recommended fill volume range is specified at a maximum (or near maximum) fill volume that is below the IPP upper limit, as predicted by the upper prediction limit. As discussed above, the application 332 may also use physiological data from a physiological sensor 338 and/or a temperature of a dialysis fluid when determining the recommended fill volume range.

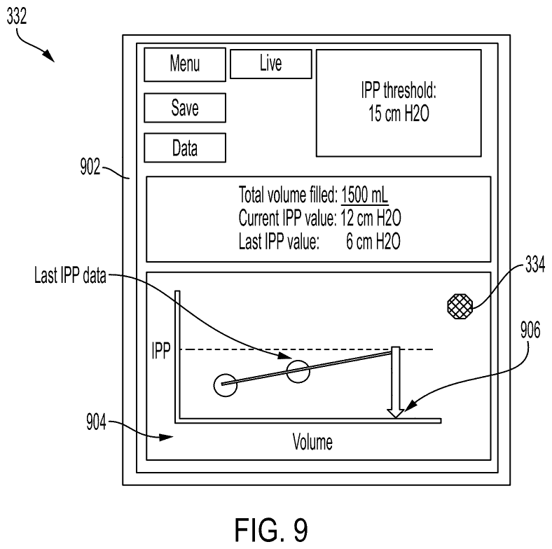

FIG. 9 is a diagram of a display interface 902 of the application 332 showing information indicative of the patient model 334, according to an example embodiment of the present disclosure. The display interface 902 includes a section that specifies an IPP upper threshold for a patient of 15 cmH₂O, which may be determined based on a patient age, gender, weight, height, health, etc. The display interface 902 also includes a section that shows a correlation of a current data point, which includes a total volume filled of 1500 mL, a current IPP measurement output data value of 12 cmH₂O, and a previous IPP measurement output data value of 6 cmH₂O. The display interface 902 further includes a patient model section 904 that illustrates previous and current data points for the patient model 334. In this example, two IPP-IPV data points have been acquired, as shown on the graph. The dashed line corresponds to the upper IPP threshold of 15 cmH₂O. The patient model section 904 also includes a recommended fill volume 906, which corresponds to an intersection of a linear extrapolation of the data points with the upper IPP threshold. In some embodiments, the recommendation 906 may be determined taking into account an upper prediction limit of the data points, a temperature of the dialysis fluid, and/or physiological data received from a sensor 338 or noted by a clinician.

Returning to FIG. 8, the example procedure 800 continues when the application 332 is configured to determine when a full prescribed volume has been reached (block 822). If a full fill volume has not been reached, the application 332 prompts the clinician or transmits an instruction to the PD machine 302 to unclamp the first fluid line 308 (block 824). The example application 332 prompts the clinician or transmits an instruction to the PD machine 302 to continue filling for a next interval or iteration until a specified volume is pumped and returns to block 806 when the specified volume is reached.

When the full fill volume has been reached, the application 332 determines a fill phase of a PD cycle is complete and begins a dwell phase (block 826). In some examples, the application 332 prompts a clinician to press a fill complete button on the PD machine 302 or the computer 330 (block 828). The application 332 then filters, removes noise, or otherwise processes the IPP measurement output data corresponding to a dwell (block 830). The application 332 updates the patient model 314 with a data point corresponding to the dwell, where the IPP-IPV (full fill volume) is associated with a time of the dwell (block 832). The dwell data point may show how IPP increases or decreases over time during a dwell as ultrafiltration accumulates, and may change a slope of a regression through the data points already collected, thereby providing an ultrafiltration buffer. In some instances, the application 332 may average the IPP measurement output data over a breathing cycle of the patient as part of the analysis. This averaging provides a stable IPP measurement (block 834). Additionally or alternatively, the application 332 analyzes a stream of IPP measurement output data to determine when the pressure measurements have stabilized. The application 332 then uses the current patient model 334 to determine and/or output a recommended fill range (block 836). The example procedure 800 then ends. In some embodiments, the patient model 334 may be transmitted to the treatment server 402 after the PD onboarding procedure is finished for determination of a PD prescription for the patient.

FIG. 10 is a diagram of the display interface 902 of the application 332 showing information indicative of the patient model 334 after a complete PD cycle, according to an example embodiment of the present disclosure. In this example, a third data point is collected for a full fill, which corresponds to 2700 mL of dialysate. Further, in this example, the upper IPP threshold is determined to be 20 cmH₂O. In some embodiments, the upper threshold may be increased or decreased at the clinician's discretion based on a patient's response (e.g., comfort/tolerance) to accommodate different fill volumes. For instance, initial patient registration data may indicate the upper IPP threshold should be 14 cmH₂O. However, the fill volume analysis may be indicative that the upper IPP threshold for the patient is actually 15.5 cmH₂O.

In this example, the full fill volume of 2700 mL corresponds to IPP measurement output data of 24 cmH₂O, which exceeds the 20 cmH₂O upper threshold, as shown in a data section 1002 of the interface 902. The application 332 performs a linear regression for the patient model 334 using the data points, including the final data point. The application 332 then determines a recommended fill volume range 906 that is located at the intersection of the linear regression curve and the 20 cmH₂O limit. The application 332 may determine a beginning of the range 906 corresponding to 80% or 90% of the maximum fill volume, which relates to a minimal amount of fluid for performing an adequate removal of waste and toxins from the peritoneal cavity of the patient.

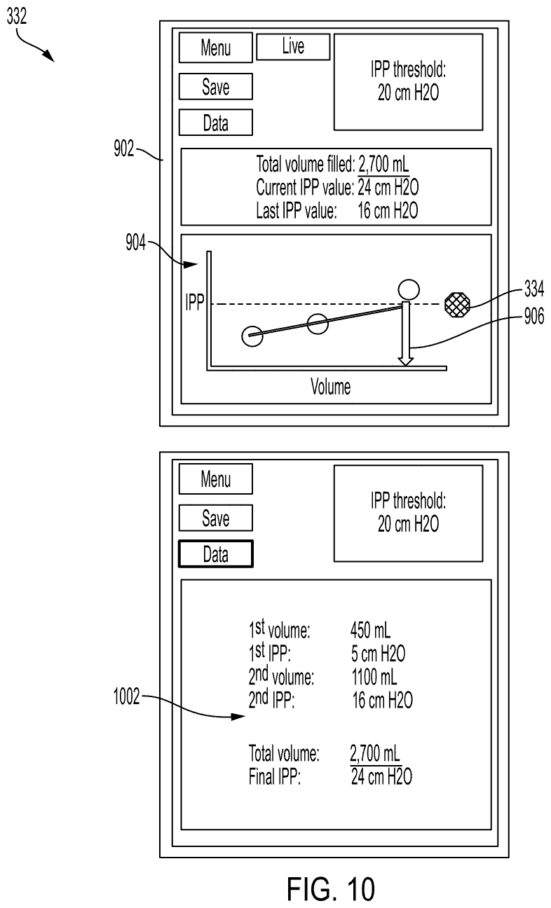

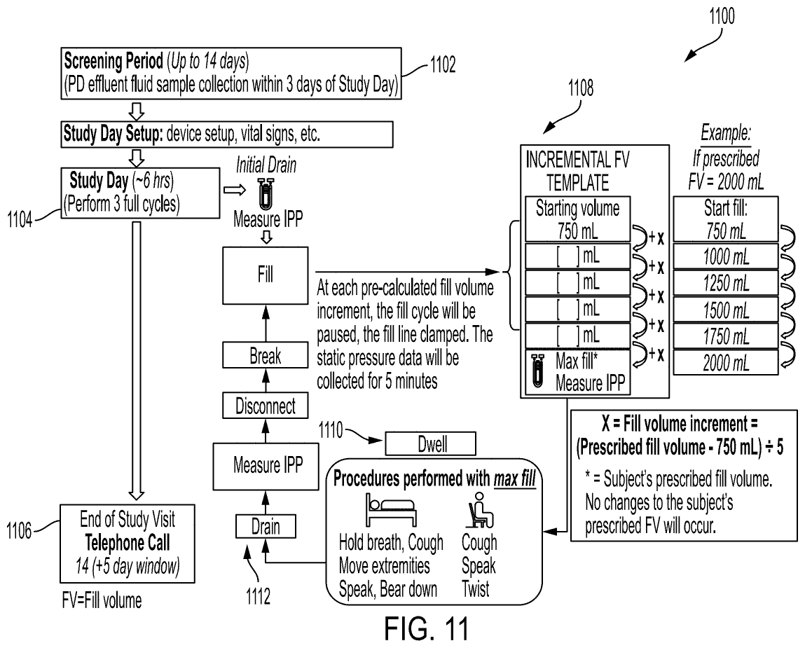

FIG. 11 is a diagram illustrative of a protocol 1100, at least a part of which is performed by the application 332 to implement a PD therapy to capture IPP data points, according to an example embodiment of the present disclosure. In the illustrated example, the protocol 1100 includes three components, including a screening component 1102, a study component 1104, and a post-study component 1106. During the screening component 1102, an effluent sample from a patient is obtained to determine peritoneal transfer characteristics. In some embodiments, a PET procedure is performed. Further, during the screening component 1102, the application receives patient co-morbidities including cardiac disease, medications taken during a 7-day window prior to evaluation, cause of kidney failure, dialysis vintage, PD vintage, prior surgical catheter revisions, peritonitis history, hernia history, etc. In some examples, an initial upper IPP threshold is determined. During the study component 1104, a patient model 334 is created using measured IPP-IPV correlations. Finally, during the post-study component 1106, a clinician follows up with a patient to ensure the prescribed PD parameters are not adversely affecting a patient, including discomfort from a high IPP.

For the study component 1104, the application 332 is configured to obtain initial IPP measurement output data after an initial drain of the patient's peritoneal cavity. The application 332 then performs a number of fill iterations during a fill phase 1108. The number of iterations is determined to be five. A fill amount per iteration is determined such that 750 mL of dialysate is provided during a first iteration. A max fill volume is determined. The fill volume of the first iteration is subtracted from the maximum fill volume and divided by the number of iterations to determine a fill amount per iteration. For a max fill target volume of 2000 mL, this corresponds to a fill iteration of 250 mL.

As shown in FIG. 11, for each iteration, the application 332 causes a specified fill volume to be provided to the patient. After the target fill volume has been reached, the application 332 causes the filling to pause and a clamp or other device to cut off pressure from the PD machine 302. After a certain time duration to enable the patient's peritoneal cavity to settle, the application 332 records IPP measurement output data from the sensor 314, which is correlated with the current total fill volume to generate a data point for the patient model 334. In some embodiments, the application 332 prompts or receives information indicative of a patient's tolerance or comfort level to help determine a maximum patient tolerance or comfort for a known IPV, which is correlated to IPP. After all iterations have been completed at the patient is at max fill, final fill IPP measurement output data is correlated to the max fill volume.

The application 332 then records one or more IPP measurement output data points during a dwell phase 1110. The application 332 may record data points at periodic times during the dwell phase to correlate how IPP measurement output data changes over time for the max fill volume. The application 332 may also record data points during certain patient physical activity during the dwell period. The physical activity may include a patient holding their breath, coughing, moving their arms/legs, speaking, bearing down, twisting, walking, etc. The physical activity may be recorded while the patient is in a sitting position and/or when the position is a supine position.

After the dwell phase 1110, the application 332 causes the PD machine 302 to drain used dialysate from the patient during a drain phase 1112. The application 332 may record IPP measurements during drain. To do so, the application 332 may cause a drain line to be clamped or indicate to the user to manually clamp the drain line periodically. The application 332 may prompt a clinician to enter a volume of fluid that has flowed into a drain bag (by volume or weight). The application 332 subtracts the drain volume from the known fill volume. The application 332 also receives or prompts a clinician for IPP measurement output data to correlate IPV to IPP during each pause during the drain phase 1112. After the patient is drained, the application 332 records IPP measurement output data for a zero fill volume associated with the drain phase 1112 to further augment the patient model 334. In some embodiments, the application 332 performs one or more cycles of fills, dwells, and drains to collect additional data points. During the cycles, dialysates with different dextrose concentrations may be used to create patient models for different dextrose levels.

### III. Patient Model Embodiments

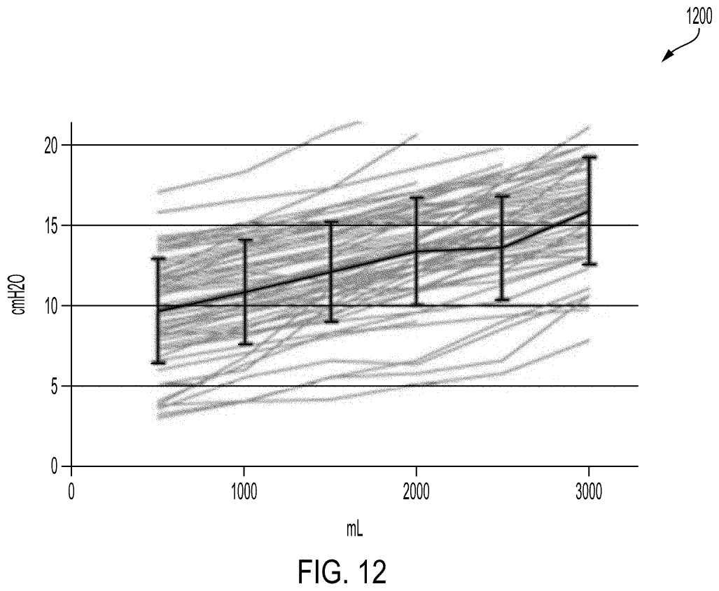

As discussed above, the patient model 334 is personalized for a patient using measured IPP-IPV correlations for multiple fill volumes. FIG. 12 is a diagram of a graph 1200 of known personalized IPP-IPV correlations for a plurality of patients, according to an example embodiment of the present disclosure. The graph 1200 shows that IPP-IPV correlations are not the same between any two patients, and vary significantly for pressure and fill volume. While the slope of each curve is roughly the same across the patients (showing a piecewise linear dependency between IPP and IPV), the slope is not consistent between each data point for the same fill volumes among the patients. The IPP-IPV correlated data in the graph 1200 shows a need for the creation of individualized patient models 334 using the procedures disclosed herein.

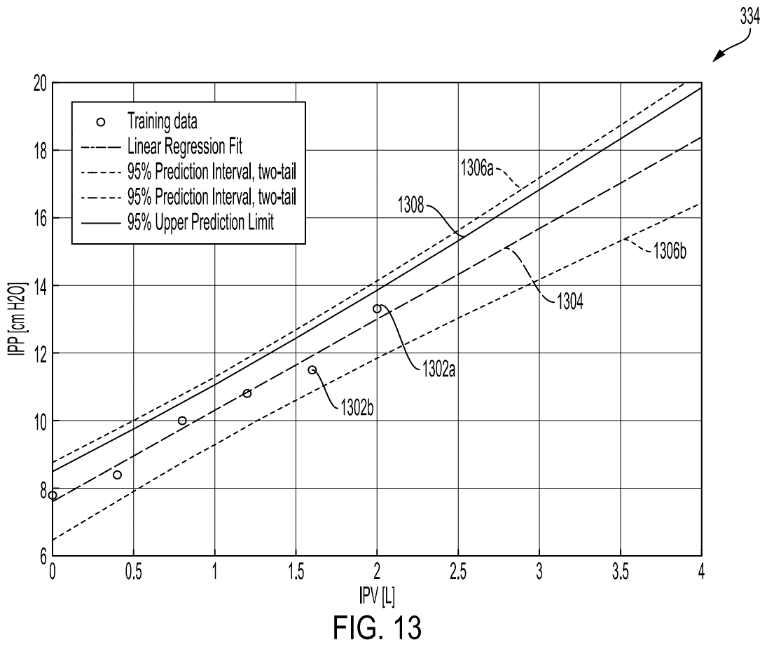

FIG. 13 is a diagram of a patient model 334, according to an example embodiment of the present disclosure. In this example, data points 1302 (identified by reference numbers 1302a and 1302b) correlating IPP to IPV for a given patient are collected by the application 332 according to the procedure discussed above in connection with FIG. 8. The application 332 is configured to perform at least a first order linear regression to determine a line or curve 1304 that is fitted to the data points 1302. In some embodiments, the application 332 may perform a regression analysis using multi-variable regression, higher order polynomials, and/or exponential functions.

The example application 332 is then configured to determine prediction intervals 1306a and 1306b for the linear regression line 1304. The prediction intervals 1306 show a range in which the actual IPP values will fall when making a new prediction with the regression line 1304, with the probability given by a confidence level (e.g., 95% in this example). In the example, the regression line 1304 and the intervals 1306 are extrapolated beyond the last data point 1302a at an IPV of 2 L. The application 332 is configured to use at least the upper prediction interval 1306a to determine a maximum IPP prediction error for each IPV. For example, as shown in FIG. 13, the IPP prediction error is +/−1.2 cmH₂O at IPV of 1 L and +/−2.2 cmH₂O at IPV of 4 L.

A one sided prediction limit 1308 is shown in FIG. 13. As shown, the upper prediction limit 1308 is preferred to the prediction interval 1306a since it enables a safer maximum fill volume to be recommended. The meaning of the upper (or one-sided) limit 1308 is that, with a confidence given by the confidence level, the resulting IPP will be below this limit for future IPP predictions.

By using the upper prediction limit 1308 for the predictive model 334, prediction confidence is added to the model compared to using the regression line 1304 as a model. The application 332 may add an IPP “safety margin” to the regression line 1304 to ensure the actual IPP will stay below the predicted value in most of the future predictions (given by the confidence level). The magnitude of the added IPP margin is dependent on the confidence level (a higher confidence increases the margin), the number of data points that the regression was based on (a larger number decreases the margin), a variance of the data points (a larger variance increases the margin), and/or an extrapolation (increases the margin).

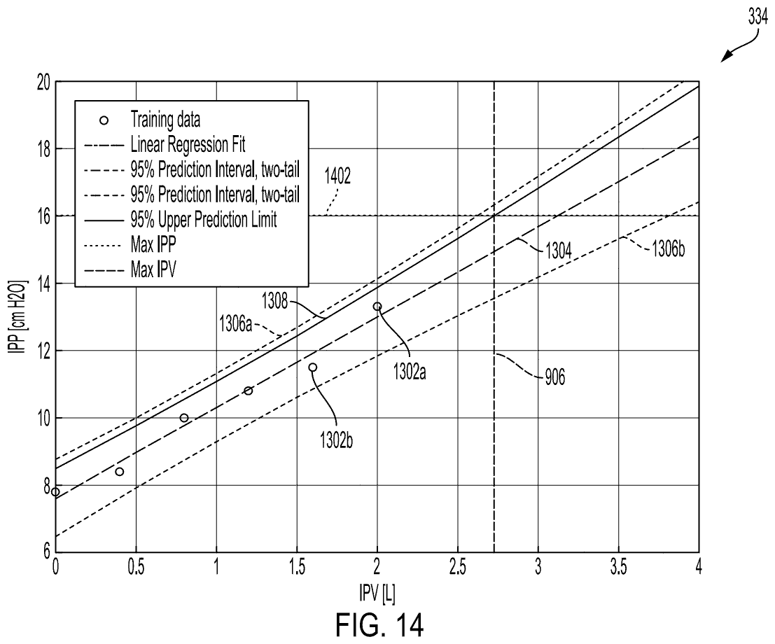

FIG. 14 is a diagram of the patient model 334 with an upper IPP limit 1402, according to an example embodiment. The upper IPP limit 1402 may be determined from the procedure 800 of FIG. 8 based on fill volumes provided to the patient. The application 332 adds the upper IPP limit 1402 to the model 334. An intersection of the upper IPP limit 1402 and the upper prediction limit 1308 corresponds to a maximum recommended fill volume 906 for the patient for a PD treatment. In this example, an upper IPP limit of 16 cmH₂O is determined, which corresponds to a maximum tolerable IPV of 2.7 L for the patient. This means that if 2.7 L of dialysate is instilled in the patient, there is a 95% probability that IPP will be below the 16 cmH₂O upper IPP limit (since the confidence level is set to 95% when the prediction model 334 was derived).

As discussed above, a patient's IPV may vary overtime depending on a number of factors. For example, IPV is a function of a residual volume in a patient's peritoneal cavity, a fill volume, a fluid composition or dextrose concentration (e.g., an osmotic gradient), a dwell time, and/or membrane transport properties (e.g., an ultrafiltration coefficient, mass transfer loss of osmotic agent(s), etc.). As such, IPP is also dependent upon the above-factors given the known correlation between IPP and IPV.

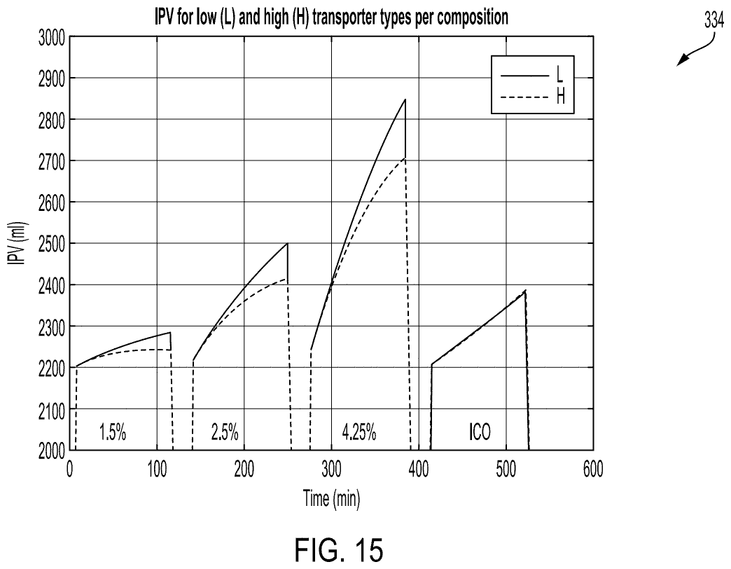

FIG. 15 is a diagram of a patient model 334 where IPV is defined as a function of time during a dwell, according to an example embodiment of the present disclosure. In the illustrated example, a first cycle is performed with dialysate having a 1.5% dextrose composition, a second cycle is performed with dialysate having a 2.5% dextrose concentration, a third cycle is performed with dialysate having a 4.25% dextrose concentration, and a fourth cycle is performed with icodextrin. The IPV was modelled using a three-pore model for low and high transporter types, thereby spanning a range of a PD population. The patient model 334 of FIG. 15 shows that IPV increases over time as ultrafiltrate accumulates in the patient's peritoneal cavity. The dialysate with the 4.25% glucose/dextrose concentration shows a volume increase by as much as 25% over time.

The example application 332 is configured, in some embodiments, to account for IPV changes over time. For instance, after a maximum volume is reached, the application 332 is configured to record IPP measurement output data periodically during a dwell phase. These additional data points ensure that a prescribed maximum fill volume remains below an upper IPP limit or threshold for a patient, even throughout a dwell phase. Alternatively, a buffer (e.g., 0.3 to 0.7 cmH₂O) may be added to the IPP measurement output data and/or subtracted from the upper IPP limit 1402 to ensure IPV changes over time do not exceed the upper IPP limit or threshold for a patient.

In some embodiments, the application 332 may provide even more precise modeling by accounting for patient transport properties, as characterized via a peritoneal equilibration test (“PET”). The application 332 may also include a transport model and consider treatment regimens including dialysate compositions and/or dwell times. The application 332 and/or the treatment server 402 may accordingly also recommend a dwell time and/or dialysate concentration as part of the electronic treatment prescription 406. In these examples, the patient model 334 is accordingly configured to predict a maximum IPV of a patient for a prescribed treatment, predict or measure a peak IPV of a patient for a prescribed treatment, determine if an alert to a physician should be generated when IPP exceeds a defined upper IPP limit, and/or determine changes to the prescription to avoid IPP peaks. For example, the treatment server 402 and/or the application 332 may determine that a maximum fill volume should be reduced by 150 mL and a dextrose concentration should be reduced to 1.5% to avoid exceeding an upper IPP limit of a patient.

While the above examples discussed the use of linear regression, it should be appreciated that the application 332 may be configured to perform a best fit of IPP-IPV data points using other mathematical relations, such as higher order polynomials, exponential functions, and/or power functions. Additionally, while the protocol 1100 and procedure 800 are described as occurring in a single day, in some embodiments they may occur over multiple days to capture patient day-to-day variability. Further, in some embodiments, the sensor 314 may be integrated with the PD machine 302 and/or the PD cassette 304 to enable the PD machine 302 to record IPP measurements during PD treatments and/or PD onboarding. In these examples, the PD machine 302 may communicate with the application 332 or the treatment server 402 for creating the patient model 334. Alternatively, the PD machine 302 may be configured to create the patient model 334. In the above embodiments, the system 300 may be used for any Automated Peritoneal Dialysis (“APD”) treatment, Continuous Ambulatory Peritoneal Dialysis (“CAPD”) treatment, or generally any renal failure replacement treatment.

### IV. IPP Measurement Integrity Verification Embodiment

In some embodiments, the application 332 is configured to verify an integrity of the IPP measurement output data from the pressure sensor 314. The application 332 is configured to generate an alert if the IPP measurement output data is indicative of an issue to prevent erroneous data from being used within the patient model 334. The alert may be indicative as to whether the pressure sensor 314 is properly fluidly connected to a patient's peritoneal cavity, for example.

The time sampled IPP measurement output data from the pressure sensor 314 contains IPP data superimposed with disturbances such as noise, heart beats, breathing, speaking, coughing, sneezing, tubing movements, and/or patient movements. An adequate fluid connection between the pressure sensor 314 and the patient may be detected by identifying vital signs in the IPP measurement output data. If no vital signs can be detected in the IPP measurement output data, it may be an indication of lost fluid connection integrity.

The application 332 may detect vital signs in the IPP measurement output data using an autocorrelation routine. The application 332 may identify a pressure signal segment of the IPP measurement output data, which is correlated with a delayed copy of itself. The delay (or phase shift) is swept to find a maximum correlation. If for example, breathing is the dominant contributor to correlation, correlation peaks may occur periodically as a function of delay time as breathing peaks coincide. This periodicity can also provide information about the breathing rate of the patient.

In some embodiments, the application 332 is configured to perform a cross-correlation of the IPP measurement output data with a synthetic signal. In this example, a pressure signal segment of the IPP measurement output data is correlated with, for example, a sine wave with the same periodicity as the breathing. To initially find the breathing rate, a correlation is calculated for several sine waves having different frequencies covering an anticipated physiological span. To initially find the phase between the signals to correlate, several phases are looped through (covering up to one breathing period). Once the breathing rate and phase have been found, the sine wave frequency range and phase range that are looped through at each algorithm execution can be drastically narrowed by the application 332.

In other embodiments, the application 332 is configured to perform a cross-correlation of the IPP measurement output data with external vital sign sensor data (e.g., using the respiratory belt 510 or the heart rate sensor 338). To initially find the phase between the pressure signal of the IPP measurement output data and the vital sign sensor data, several phases are looped through for analysis by the application 332. Once the phase has been found, the phase range looped through at each algorithm execution can be drastically narrowed by the application 332. The correlated pressure signal can include the raw signal or a version of the raw signal of the IPP measurement output data that has been filtered in a way to maximize the chance of vital sign detection by exclusively keeping the vital sign in the pressure signal (e.g., a band pass filter with a pass range of 0.1-0.5 Hz to keep breathing pulsations or a pass range of 0.8-2.5 Hz to keep heart pulsations).

The application 332 may normalize the calculated correlation with the magnitudes of both correlated signals to achieve a correlation coefficient (“CorrCoeff”) ranging from −1 to 1. Where: CorrCoeff=1: perfect correlation between correlated signals, CorrCoeff=0: no correlation between correlated signals, and CorrCoeff=−1: perfect correlation between “vertically mirrored” correlated signals. The correlation coefficient can be used as a measure of integrity probability with a high value (close to one) indicating a high probability and a value closer to zero indicating a low probability. A correlation coefficient value close to −1 suggests an inverse relationship between the signals and could possibly be used for integrity probability assessment by the application.

To assess integrity, the application 332 compares the correlation coefficient to a threshold or a range. For example, the application asserts integrity if CorrCoeff>0.5 or −0.5>CorrCoeff>0.5. Some information on integrity probability can be retrieved from the phase signal. If the phase shift where maximum correlation occurs differs from expected or historical value, it can be an indication of integrity loss.

FIG. 16 is a diagram of a graph 1600 showing an assessment of integrity of the IPP measurement output data from the in-line pressure sensor 314, according to an example embodiment of the present disclosure. Specifically, the graph 1600 shows a cross-correlation between respiratory belt and pressure signal (both band pass filtered) of the IPP measurement output data. In case of lost integrity between the pressure sensor 314 and the patient, the breathing pulsations suddenly disappear from the pressure signal. This results in a significantly lower correlation that is likely to be close to zero, and hence the integrity loss can be detected. In the illustrated example, there is good correlation between the filtered pressure signal of the IPP measurement output data and respiratory belt information, indicating the in-line sensor 314 has a good fluid coupling with the patient.

### V. Processing of the IPP Measurement Output Data Embodiment

As discussed above, the pressure sensor 314 is configured to generate and transmit a continuous or near-continuous stream of IPP measurement output data. As described below, the application 332 is configured to process the IPP measurement output data to enable a true average IPP value to be determined. The time-sampled pressure signal of the IPP measurement output data contains the IPP and superimposed disturbances such as noise, heart beats, breathing, tubing movements, and patient movements. To extract a pressure value that represents the patient's IPP from the pressure signal of the IPP measurement output data, it should be ensured that the variability from those disturbances do not alter the IPP reading.

The example application 332 may perform low-pass filtering to suppress disturbances. The use of a low-pass filter cuts off or suppresses disturbances above a certain frequency. The selected filter characteristics are a trade-off between disturbance suppression performance and speed. The cut-off frequency is selected to filter out or remove periodic vital signs (<<0.2 Hz which is a typical breathing rate). The disturbances that remain in the filtered signal have a low frequency and typically originate from patient movements and body position changes.

In some embodiments, the application 332 calculates a variance over a sliding window of the low-pass filtered signal to obtain a measure of how much low-frequent disturbances remain in the filtered signal. Low variance indicates that the filtered signal has been stable for a period with a length of the sliding window and that it is a suitable opportunity to extract an IPP value from the filtered signal. High variance on the other hand, suggests the opposite. Stable conditions that enable IPP extraction is assessed when the variance is below a predetermined variance acceptance limit. The length of the sliding variance window is configured to detect disturbances of an anticipated range of durations. A reasonable time is 20 seconds, for example.

The application 332 may also use a signal delay. If a low-frequency disturbance enters the filtered signal, there is a certain delay before the variance calculated above rises to a level where IPP extraction is deemed inadequate. This may result in an extraction of an erroneous IPP in a situation when the filtered signal level has been altered considerably by a disturbance although the variance has not yet exceeded the variance acceptance limit. If a new signal is created by delaying the filtered signal, the real-time variance signal is used for stability assessment and predicts coming disturbances in the delayed version of the filtered signal. The delay time depends on the low-pass filter characteristics and the length of the variance calculation window. The application 332 may extract an IPP value from the delayed filtered signal after the variance signal is below the variance acceptance limit.

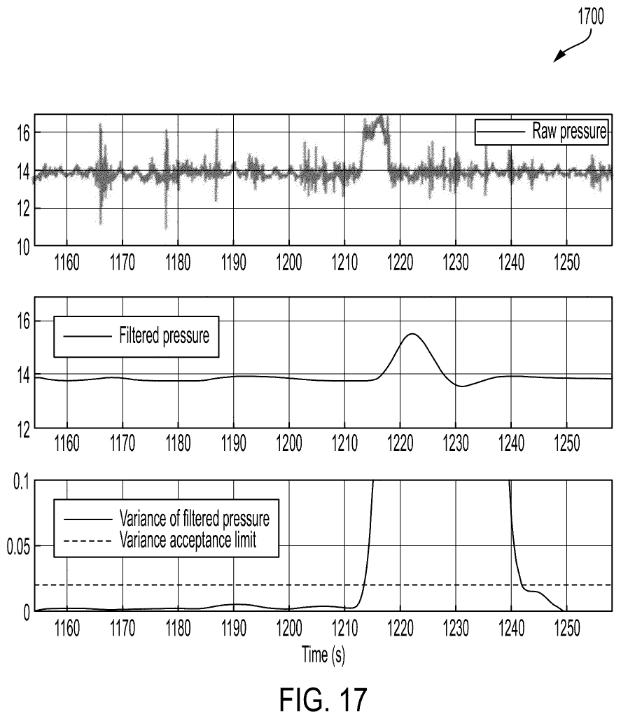

FIG. 17 is a diagram of a graph 1700 showing a raw time sampled (100 Hz) pressure signal from the IPP measurement output data. The signal includes pressure in addition to noise, heart beats, breathing, and physical disturbances. A distinct disturbance can be seen in the raw pressure sensor signal, starting at 1212 seconds and lasting for approximately 5 seconds, during which the pressure level is altered by 2 cmH₂O. A middle plot shows the raw pressure filtered with a 4th order butterworth low-pass filter with a cut-off frequency of 0.07 Hz. Noise, heart beats, breathing, and high frequency physical disturbances are removed, but the disturbance starting at 1212 seconds has not been fully suppressed by the filter and is still present in the filtered signal between 1215-1235 seconds.

The lower plot of FIG. 17 shows a 20 second sliding window variance of the filtered signal along with a variance acceptance limit to be used for assessing if stable conditions exist. As the filtered signal is stable during the first half, the variance is well below the variance acceptance limit. As the disturbance present in the filtered signal becomes contained in the variance calculation window, the variance rises steeply and soon exceeds the variance acceptance limit, thereby inhibiting IPP extraction from the filtered signal. When comparing the timing of the disturbance entry into the filtered signal and the rise of the variance signal, it can be seen that the latter occurs just prior to the former. This is a result of a 5 second delay that has been applied to the filtered pressure. By doing so, the segment where IPP extraction is deemed inadequate is effectively centered over the disturbed segment in the filtered signal.

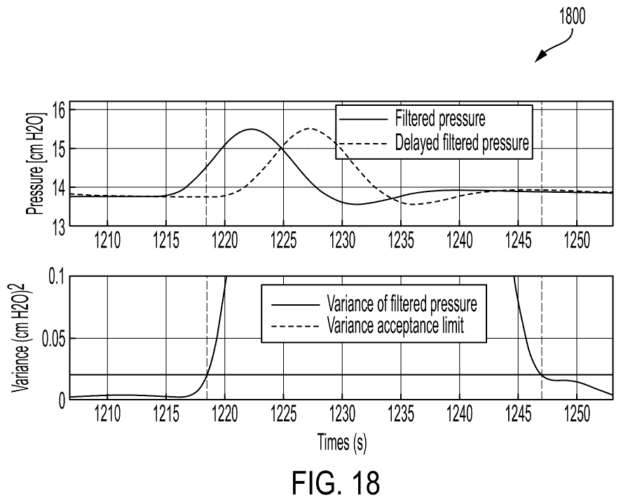

FIG. 18 is a diagram that illustrates signal delays of the signals shown in FIG. 17, according to an example embodiment of the present disclosure. In the upper plot, a low frequency disturbance enters the filtered pressure (blue) at 1215 seconds. In the lower plot, the variance begins to rise shortly thereafter and exceeds the variance acceptance limit at 1218 seconds. At 1218 seconds, the filtered pressure (blue) has rose 0.7 cmH₂O above the baseline value. This means that if IPP had been extracted just before 1218 seconds, an error of +0.7 cmH₂O would be added. If instead the 5 seconds delayed filtered pressure (red) is used for IPP extraction at 1218 seconds, no error is introduced.

In some embodiments, the low-pass filter can be replaced with filters such as median filter or moving average. Further, the variance stability measure can be replaced with sliding standard deviation or sliding max-min range. Moreover, additional constraints for IPP extraction can be added such as stability deemed for a certain amount of time. To avoid too long IPP extraction procedures, the IPP extraction constraints may be adapted depending on how long time IPP extraction has lasted. The application 332 may perform a plausibility check on the extracted IPP to identify, for example, an outlier compared to previous data.

In addition to above, a measure of stability could be used by the application 332 to assess the extent to which disturbances other than periodic vital signs (breathing and heart) are present in the signal. This may include autocorrelation of the pressure signal, cross-correlation of the pressure signal with synthetic signals (e.g. sine waves of different frequencies), and/or cross-correlation of the pressure signal with external vital sign sensor (e.g. respiratory belt or heart rate sensor) as discussed above in connection with FIG. 16. The correlated pressure signal could be the raw signal or a version of the raw signal that has been filtered in a way to keep the vital signs to be correlated (e.g. a band pass filter with a pass range of 0.1-0.5 Hz to keep breathing pulsations or a pass range of 0.8-2.5 Hz to keep heart pulsations).

### VI. Continuous Ambulatory PD (“CAPD”) Embodiment

The system 300 discussed above primarily includes the PD machine 302. In other embodiments, IPP-IPV correlations may be determined using a manual exchange, such as a CAPD configuration. FIG. 19 is a diagram of the system 300 configured for CAPD, according to an example embodiment of the present disclosure. In the illustrated example, the PD machine 302 is replaced with the fluid container 306 or a heated fluid container, which is directly connected to the first fluid line 308 for priming and filling a peritoneal cavity of a patient. As shown, the fluid container 306 is placed above a head height of the patient (e.g., within 1000 mm of a patient's head height) to enable gravity to move a dialysis fluid from the container 306 through the patient lines 308 and 316 to the PD transfer set 318 and into the peritoneal cavity. The head height range corresponds to a point of entry into the patient relative to a vertical middle of the fluid container 306.

FIG. 19 also shows a clamp 1902 (e.g., a valve) that is placed on the first fluid line 308 between the sensor 314 and the fluid container 306. The clamp 1902 needs to be at a same level compared to a height of the sensor 314 to prevent additional head height from impacting pressure measurements. The clamp 1902 may be actuated manually by a clinician. Alternatively, the clamp 1902 may include a motor or relay for automatic actuation based on instructions from the application 332. In these instances, the clamp 1902 may be wired or wirelessly coupled to the computer 330 to receive instructions.

The example clamp 1902 is actuated to occlude the first fluid line 308 when IPP measurements are recorded. In some embodiments, the clamp 1902 is closed and the application 332 waits for fluid in the peritoneal cavity to settle a few seconds to a few minutes before an IPP measurement is performed. After an IPP-IPV data point is recorded, the application 332 causes the clamp 1902 to open, thereby permitting additional fluid to flow from the container 306.

During a fill phase, a volume of fluid provided to the patient may be noted by a clinician based on a fluid level in the container 306. A difference from a previously reported fluid level corresponds to an amount provided to the patient. Alternatively, the fluid container 306 may be placed on or hung from a weight scale 1904. A clinician may note a weight when an IPP measurement is performed by the sensor 314, and enter the weight into the computer 330. In some instances, the weight scale 1904 may be communicatively coupled to the computer 330 and transmit weight data when requested by the application 332 or prompted via a clinician. The application 332 may determine a weight difference from a previous IPP measurement or from an initial fluid container weight to calculate a volume provided to the patient.

The embodiment of FIG. 19 also shows a drain container 1906 placed on a weight scale 1908 (which could also be embodied by hanging from a scale). In some embodiments, the scale 1908 is the same scale as the scale 1904. Further the drain container 1906 may be the same fluid container as fluid container 306, but moved to a floor or lower level after a fill is complete. As shown in FIG. 19, after a fill is complete, the clamp 1902 is closed and the first patient line 308 is disconnected from the fluid container 306 and connected to the drain container 1906 (or the container 306 is moved to the floor), which is located on a floor or any location lower than an entry point into a patient's peritoneal cavity.

After a dwell phase, the clamp 1902 is opened, permitting used or spent dialysis fluid to flow from the peritoneal cavity into the drain container 1906. The clamp 1902 may be closed periodically during drain to enable IPP measurements to be made. A weight of the drain container 1906 is transmitted or entered into the application 332 to provide a correlated IPP-IPV data point. In some embodiments a volume of fluid in the drain container 1906 is entered into or otherwise communicated to the computer 330. This drain procedure continues until the patient is completely or near completely drained.

VII. Dialysis Machine Embodiments

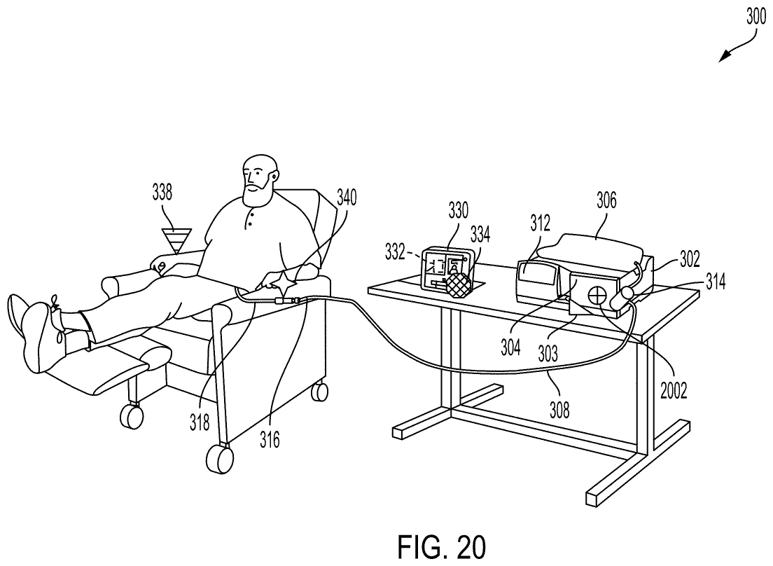

In the embodiments described above, the sensor 314 is placed in-line between the first fluid line 308 and the second fluid line 316. In other embodiments, the sensor 314 may be integrated with the PD machine 302 (e.g., a PD cycler). FIG. 20 is a diagram of the system 300 of FIG. 3 with the sensor 314 included within the PD machine 302, according to an example embodiment of the present disclosure.

In this example, the sensor 314 may include any pressure sensor configured to measure a fluid pressure within the fluid lines 308 and 316, which is indicative of pressure in a patient's peritoneal cavity. The sensor 314 may be provided in-line within the PD machine 302, located downstream from a clamp 2002 (e.g., a valve) and upstream from a to-patient tube port. When engaged, the clamp 2002 is configured to isolate any pressure within the PD machine 302 from the sensor 314 such that the only pressure in the fluid lines 308 and 316 is from the patient's peritoneal cavity. In some embodiments, the sensor 314 and/or the clamp 2002 are operable with sections of the cassette 304. The sections may include a flexible membrane or tube. For example, the pressure sensor 314 may be operable with a first section of the cassette 304 to detect a fluid pressure imparted by the fluid section on the sensor 314. Additionally, the clamp 2002 may include an actuator that imparts a force on a second section of the cassette 304 to occlude fluid flow through the cassette.

It should be appreciated that the PD machine 302 is placed at a head height with respect to a fluid entry port of the patient. This placement ensures the pressure measurement performed by the sensor 314 accounts for additional pressure from a head height contributed by the fluid lines 308 and/or 316 during the initial setup and calibration process. In some embodiments, the PD machine 302 may be raised or lowered based on a position of a patient, where a difference in head height can be accounted for during a calibration process. Moreover, a plurality of sensors 340 (e.g., multi-axis accelerometers) may detect and provide indications of the relative motion between the sensor and the patient. The sensor 340 may be wired or wirelessly coupled to the computer 330. The sensors 340 can be placed in a way that a new calibration is needed to compensate for a new head height difference between the patient and the pressure sensor.

The clamp 2002 and the sensor 314 communicate with a processor of the PD machine 302. The processor receives IPP measurement output data from the sensor 314 and instructs the clamp 2002 to open or close. The processor may be communicatively coupled to the computer 330 via a wired or wireless connection. The processor transmits the IPP measurement output data to the application 332 for processing, as discussed above. In these embodiments, the application 332 may transmit instructions to the processor of the PD machine 302, which cause the PD machine to start or stop pumping dialysis fluid to the patient during a fill phase. The instructions may also cause the PD machine 302 to close or open the clamp 2002. The instructions may further cause the PD machine 302 to transmit the IPP measurement output data from the sensor 314. In this manner, the application 332 is configured to provide automated control of the PD machine to automatically determine an IPP-IPV correlation for a patient.

As discussed above, IPP at IPV=0 (recorded directly after drain (IPP0)) is a measure of the intercept of the IPP-IPV correlation and could be determined at a clinic (or possibly at home with the PD machine 302). The delta IPP resulting from filling the patient can be measured by the PD machine 302 regardless of vertical alignment of the patient and the PD machine 302 if the pressure is measured directly after drain (P0) and after a volume has been filled (P). Delta IPP is calculated as P−P0, and thus IPP can be calculated as IPP0+delta IPP for any fill volume. This can be used to re-establish the slope of the patient's IPP-IPV correlation regularly in the home (but with IPP0 established at a clinic) as well as for more or less continuous IPP monitoring throughout APD treatments.

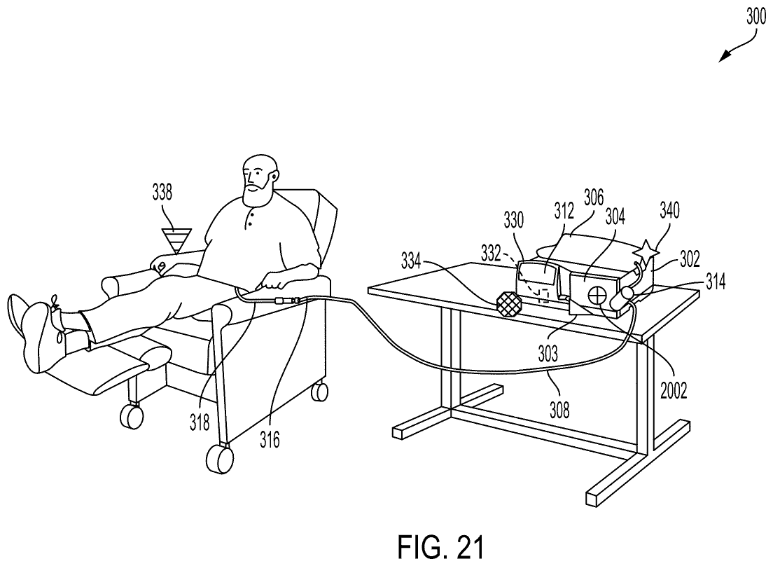

FIG. 21 is a diagram of an embodiment where the computer 330, including the application 332, is included in the PD machine 302, according to an example embodiment of the present disclosure. The computer 330 may be embodied by a processor of the PD machine 302 or a separate controller. Further, the application 332 may be executed by the internal processor or controller of the PD machine 302 to perform the operations discussed herein.

The inclusion of the computer 330 with the PD machine 302 enables the application 332 to direct operation of PD cycles including starting and stopping pumps for patient fills and patient drains. Further, the use of the internal sensor 314 and optionally the sensor 340 enables the application 332 to record IPV-IPP data points without needing an external connection to another device or sensor. However, it should be appreciated that the PD machine 302 may be communicatively coupled to the treatment server 402 via the network 404, as shown in FIG. 4. For instance, the application 332 may transmit the patient model 334 and/or related data points to the server 402 for efficiently determining an optimized and personalized PD treatment for a patient.

### VIII. Conclusion

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. An intraperitoneal pressure (“IPP”)-intraperitoneal volume (“IPV”) correlation system comprising:
a fluid container including dialysate;
a dialysis machine fluidly coupled to the fluid container;
an in-line pressure sensor fluidly coupled to the dialysis machine and configured to transmit IPP measurement output data;
a catheter fluidly coupling the in-line pressure sensor to a peritoneal cavity of a patient; and
a computer communicatively coupled to the in-line pressure sensor, the computer including an application stored in a memory device, which when executed by a processor of the computer, causes the computer to:
determine or receive an indication of a maximum fill volume of the dialysate to be pumped into the peritoneal cavity of the patient for a fill or a drain phase of a dialysis cycle,
determine a plurality of iterations to achieve the maximum fill volume and a volume of the dialysate to be pumped for each of the iterations,
for each iteration
cause the dialysis machine to pump the dialysate to the peritoneal cavity of the patient based on the determined volume for that iteration,
cause the dialysis machine to stop pumping the dialysate when the determined volume is reached,
cause at least one clamp or valve to be actuated to isolate pressure from the dialysis machine,
record IPP measurement output data from the pressure sensor,
record or determine a total accumulated fill volume,
create a data point corresponding to a correlation between the IPP measurement output data and the total accumulated fill volume, and
release the at least one clamp or valve to enable pumping of the dialysate for a next iteration, and
after the plurality of iterations are complete, create a patient model that provides a personalized correlation between IPP measurement output data and total accumulated fill volumes up to the maximum fill volume using the created data points.

2. The system of claim 1, wherein execution of the application is further configured to cause the computer to:
after the fill phase of the dialysis cycle, cause the dialysis machine to perform a dwell phase;
record periodic measurements of IPP measurement output data from the pressure sensor during the dwell phase; and
create data points for the patient model corresponding to a correlation between the periodic measurements of IPP measurement output data and the maximum fill volume over a time duration corresponding to the dwell phase.

3. The system of claim 2, wherein the application is configured to adjust the patient model using information indicative of ultrafiltration accumulation in the peritoneal cavity of the patient during the dwell phase, and
wherein the information indicative of ultrafiltration accumulation in the peritoneal cavity of the patient during the dwell phase is used to determine an optimal ultrafiltration time for a PD treatment dwell phase.

4. The system of claim 1, wherein the application is further configured to use the data points of the patient model to:
determine a regression curve through the data points; and
determine prediction intervals for the regression curve specifying a range in which IPP measurement output data will fall when making a new prediction.

5. The system of claim 4, wherein the application is further configured to use the regression curve to determine an upper prediction limit.

6. The system of claim 5, wherein the application is further configured to:
determine or receive information indicative of an upper IPP limit for the patient; and
display in the patient model the upper IPP limit in relation to the upper prediction limit to enable a maximum fill volume for a peritoneal dialysis treatment for the patient to be determined.

7. The system of claim 5, wherein the application is further configured to:
determine or receive information indicative of an upper IPP limit for the patient; and
determine a recommended maximum fill volume range having an upper bound corresponding to an IPV at an intersection of the upper IPP limit and the upper prediction limit.

8. The system of claim 5, further comprising a treatment server communicatively coupled to the application, the treatment server configured to:
receive, from the application, the patient model with the regression curve, the prediction intervals, and the upper prediction limit;
determine or receive information indicative of an upper IPP limit for the patient; and
determine a recommended maximum fill volume corresponding to an IPV at an intersection of the upper IPP limit and the upper prediction limit.

9. The system of claim 8, wherein the treatment server is configured to:
store the recommended maximum fill volume to an electronic treatment prescription; and
transmit the electronic treatment prescription to a dialysis machine associated with the patient.

10. The system of claim 9, wherein the upper IPP limit is based on at least one of a patient age, a patient height, a patient weight, a patient health, a time on dialysis, a patient position, or a patient gender.

11. The system of claim 1, wherein the dialysate has a first concentration of dextrose or glucose and the dialysis cycle is a first dialysis cycle, and wherein the application is configured to perform at least one addition dialysis cycle for dialysates having at least one different concentration of dextrose or glucose to obtain additional data points for the patient model.

12. The system of claim 1, wherein the dialysis cycle is a first dialysis cycle, and
wherein the application is configured to perform at least one addition dialysis cycle to obtain additional data points for the patient model.

13. The system of claim 1, wherein the dialysis machine is a peritoneal dialysis machine.

14. The system of claim 1, wherein the in-line sensor is positioned at a midline of the patient.

15. The system of claim 14, wherein the application is configured to output a pressure output from the pressure sensor to enable raising or lowering of the pressure sensor for positing at the midline of the patient.

16. The system of claim 1, wherein the application is configured to zero the pressure sensor to atmospheric pressure.

17. The system of claim 1, further comprising a physiological sensor configured to measure a physiological parameter of the patient, wherein the application is further configured to:
for each iteration, receive data indicative of the physiological parameter of the patient from the physiological sensor; and
associate the physiological parameter to the corresponding data point of the patient model created during the iteration for subsequent adjustment of at least one of a regression curve, prediction intervals, or an upper prediction limit determined from the patient model.

18. The system of claim 1, further comprising a physiological sensor configured to measure a physiological parameter of the patient, wherein the application is further configured to:
for each iteration, receive data indicative of the physiological parameter of the patient from the physiological sensor; and
associate the physiological parameter to the corresponding patient model to address changes in intraperitoneal volume due to ultrafiltration and changes in a transport capacity specific to a peritoneal membrane of the patient.

19. The system of claim 1, wherein the application is further configured to determine the maximum fill volume by receiving information indicative of the maximum fill volume, and
wherein the application is further configured to determine the plurality of iterations by receiving information indicative of the plurality of iterations.

20. The system of claim 1, wherein the computer is communicatively coupled to the dialysis machine, and
wherein the application is further configured to transmit instructions to the dialysis machine to cause the dialysis machine to pump the dialysate to the peritoneal cavity of the patient and cause the dialysis machine to stop pumping the dialysate when the determined volume is reached.

21. The system of claim 1, wherein the computer is communicatively coupled to the dialysis machine, and
wherein the application is further configured to receive the total accumulated fill volume from the dialysis machine.

22. The system of claim 1, wherein at least one of the in-line pressure sensor or the computer is integrated with the dialysis machine.

23. The system of claim 1, wherein the application is further configured to perform an integrity test by:
filtering the IPP measurement output data;
receiving patient movement or breathing data;
comparing the filtered IPP measurement output data to the patient movement or breathing data; and
providing an indication of an issue with the IPP measurement output data after detecting a deviation in a correlation between the filtered IPP measurement output data and the patient movement or breathing data.

24. The system of claim 1, wherein the IPP measurement output data includes a pressure signal, and the application is further configured to:
sample the IPP measurement output data to extract the recorded IPP measurement output data.
